(19) United States
(12) Patent Application Publication
(10) Pub. No.: US 2018/0256912 A1
(43) Pub. Date: Sep. 13, 2018

(54) METHOD FOR MONITORING TREATMENT OF NEUROPSYCHIATRIC DISORDERS

(71) Applicant: THE REGENTS OF THE UNIVERSITY OF CALIFORNIA, Oakland, CA (US)

(72) Inventors: Andrew Leuchter, Los Angeles, CA (US); Ian Cook, Los Angeles, CA (US); Juliana Corlier, Los Angeles, CA (US); Aimee Hunter, Santa Monica, CA (US)

(21) Appl. No.: 15/914,801

(22) Filed: Mar. 7, 2018

Related U.S. Application Data
(60) Provisional application No. 62/468,118, filed on Mar. 7, 2017.
(60) Provisional application No. 62/580,007, filed on Nov. 1, 2017.

(51) Int. Cl. A61N 2/00; A61B 5/048; A61B 5/00; A61B 5/0478

(52) U.S. Cl. CPC A61N 2/006; A61B 5/048; A61B 5/0478; A61B 5/7246; A61B 5/4848; A61B 5/7242

(57) ABSTRACT

The present invention provides methods for monitoring neuropsychiatric treatment of depression and other disorders. The methods monitor the progress of neuropsychiatric treatment by examining electrical oscillations in the brain as measured by quantitative electroencephalography (qEEG). The methods are useful in predicting and guiding the outcome of neuropsychiatric treatment.

### CROSS-REFERENCE TO RELATED APPLICATIONS

This application claims priority to U.S. Provisional Patent Application No. 62/468,118, filed Mar. 7, 2017, and to U.S. Provisional Patent Application No. 62/580,007, filed Nov. 1, 2017, the contents of each being incorporated by reference herein in their entirety.

### BACKGROUND OF THE INVENTION

Depression is the second leading cause of medical disability in the United States and affects tens of millions of people each year. Neuromodulation therapies such as deep brain stimulation (DBS) and repetitive transcranial magnetic stimulation (rTMS) offer the potential to relieve symptoms in patients who are unresponsive to medication. Neuromodulation therapies are coming into widespread use. However, there currently are no means to monitor brain function to predict outcomes of TMS treatment. The state of the art is “watchful waiting” and monitoring patients' symptoms to determine if and when they will respond.

There is a need in the art for an improved method for monitoring neuropsychiatric treatment of depression and other disorders to predict and guide treatment. The present invention meets this need.

### SUMMARY OF THE INVENTION

In one aspect, the present invention relates to a non-transitory computer-readable medium with instructions stored thereon, that when executed by a processor, determines a correlation coefficient of the shift in pre- and post-transcranial magnetic stimulation (TMS) treatment between a pair of EEG electrodes positioned on at least two sites of a subject's brain, by performing the steps comprising: measuring a subject's pre-rTMS power spectrum at an EEG electrode of interest; calculating relative power from the subject's pre-rTMS power spectrum measurements at the EEG electrode of interest; measuring a subject's post-rTMS power spectrum at the EEG electrode of interest; calculating a relative power from the subject's post-rTMS power spectrum measurements at the EEG electrode of interest; obtaining a correlation coefficient R

R=corr((S_{t1,F}−S_{t0,F}),(S_{t1,S}−S_{t0,S})) (Eq.1)

wherein S_{ti,Loc}=relative power of the spectrum measurements; tᵢ=t₀ for pre-rTMS; tᵢ=t₁ for post-rTMS; Loc=F for first brain site; and Loc=S for second brain site.

In one embodiment, the stored instructions further comprise the step of determining a dynamic response (DR) value from the correlation coefficients of a selection of electroencephalography (EEG) electrode pairs, wherein the DR value is defined according to:

$$DR = \frac{{\sum }_{i = 1}^{n} ( {R}_{i} )}{n} \qquad ( Eq 2 )$$

wherein n=the number of selected EEG electrode pairs.

In one embodiment, the power spectrum is measured in the alpha band, the beta band, the gamma band, the delta band, or the theta band. In one embodiment, the at least two sites of the subject's brain are selected from the group consisting of the left or right side of: the frontal lobe, the central lobe, the temporal lobe, the parietal lobe, the occipital lobe, the motor cortex, the premotor cortex, the prefrontal cortex, the somatosensory cortex, the posterior parietal cortex, the visual cortex, the auditory cortex, the temporal cortex, the frontal gyms, the postcentral gyms, the lateral occipital gyms, the temporal gyms, the Brodmann areas, the cuneus, the precuneus, and combinations thereof. In one embodiment, the two sites of the subject's brain are the frontal lobe and the parietal lobe.

In one embodiment, the at least two sites of the subject's brain form part of a brain network selected from the group consisting of: the frontoparietal control network (FCN); the default mode network (DMN); the salience network (SN); the dorsal attention network (DAN); the ventral attention network (VAN); the basal ganglia network (BGN); the limbic network (LN); the somatomotor network (SMN); the visual network (VN); the frontoparietal network (FPN); the anterior insula network (AIN); the executive control network (ECN); the executive attention network (EAN); the medial visual network (MVN); the lateral visual network (LVN); the cerebellar network (CBLN); the auditory network (AN); the task positive network (TPN); and the self-referential network (SRN)

In one embodiment, the dynamic response is the alpha dynamic response (αDR), the beta dynamic response (βDR), the gamma dynamic response (γDR), the delta dynamic response (ΔDR), or the theta dynamic response (θDR). In one embodiment, the power spectrum is measured at a peak individual alpha frequency (IAF) value in the range between 2 and 20 Hz. In one embodiment, the power spectrum is measured between an IAF band between 2 Hz below the IAF value and 2 Hz above the IAF value.

In one embodiment, the subject's pre-TMS power spectrum at the EEG electrode of interest is measured immediately before the administration of a TMS treatment session. In one embodiment, the subject's post-TMS power spectrum at the EEG electrode of interest is measured one minute after administration of a TMS treatment session.

In one embodiment, the selection of EEG electrode pairs is the four EEG electrode pairs: Fp1-Pz, Fpz-Pz, F3-Pz, and F5-Pz. In one embodiment, the determined αDR value is between −1 and 1, such that a value closer to 1 indicates the subject has greater responsiveness to the rTMS treatment and a value closer to −1 indicates the subject has lesser responsiveness to the rTMS treatment.

In another aspect, the present invention relates to method of monitoring treatment of a neuropsychiatric disorder in a subject, comprising the steps of: recording pretreatment quantitative electroencephalogram (qEEG) measurements of the subject as a function of relative power over a frequency range between 2 and 20 Hz; treating the subject; recording posttreatment qEEG measurements of the subject as a function of relative power over a frequency range between 2 and 20 Hz; and rating the effectiveness of the treatment based on the change in the frequency of peak relative power between pretreatment and posttreatment qEEG measurements.

In one embodiment, the change between pretreatment and posttreatment qEEG measurements shows an increased and narrowed mean frequency primary peak and the emergence of a higher frequency secondary peak, indicating the treatment is highly effective in treating the subject.

In one embodiment, the change between pretreatment and posttreatment qEEG measurements shows a narrowed mean frequency primary peak and the emergence of one or more higher frequency secondary peaks, indicating the treatment is moderately effective in treating the subject.

In one embodiment, the change between pretreatment and posttreatment qEEG measurements shows a decreased mean frequency primary peak and the emergence of one or more higher frequency secondary peaks, indicating the treatment is slightly effective in treating the subject.

In one embodiment, the change between pretreatment and posttreatment qEEG measurements shows little to no change in the mean frequency primary peaks and secondary peaks, indicating the treatment is not very effective in treating the subject.

In one embodiment, the neuropsychiatric disorder is selected from the group consisting of: major depressive disorder (MDD), anxiety, post-traumatic stress disorder (PTSD), obsessive compulsive disorder (OCD), and Parkinson's disease.

In one embodiment, the treatment is selected from the group consisting of: repetitive transcranial magnetic stimulation (rTMS), deep brain stimulation, and transcranial direct current stimulation.

In one embodiment, the pretreatment and posttreatment qEEG measurements are recorded between 7.5 and 14 Hz.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 depicts graphs showing the quantitative electroencephalogram (qEEG) activity of a subject with Major Depressive Disorder (MDD) before (top row) and after (bottom row) repetitive transcranial magnetic stimulation (rTMS) treatment. The subject entered remission at the conclusion of treatment, a complete resolution of symptoms.

FIG. 2 depicts graphs showing the qEEG activity of a subject with MDD before (top row) and after (bottom row) rTMS treatment. The subject had a response to treatment, a 50% reduction in depressive symptoms.

FIG. 3 depicts graphs showing the qEEG activity of a subject with MDD before (top row) and after (bottom row) rTMS treatment. The subject had improvement to treatment but experienced a less than 50% reduction in depressive symptoms.

FIG. 4 depicts graphs showing the qEEG activity of a subject with MDD before (top row) and after (bottom row) rTMS treatment. The subject showed a worsening of symptoms with treatment.

FIG. 5A through FIG. 5D depict the shift in mean frequency for the first six minutes following the end of a first TMS treatment for three patients: FIG. 5A is a patient who entered remission following six weeks of treatment (i.e. a complete resolution of depressive symptoms); FIG. 5B is a patient who was a TMS responder (i.e., a patient with substantial improvement, but not remission of depression); and FIG. 5C and FIG. 5D are patients who were non-responders to rTMS treatment. Each minute following treatment is shown in a separate row from top to bottom, with the shift in mean peak frequency indicated by the blue lines (an upward deflection represents an increase in relative power at the frequency, and a downward deflection a decrease). The average shift in mean peak frequency is displayed in four brain regions from the left column to the right column: frontal, central, temporal, and parieto-occipital regions. In the left column (frontal lobe) of FIG. 5A, it is clear that the patterns of mean peak frequency shifts seen in the frontal region change from minute to minute following the end of TMS treatment, with shifts in mean frequency that evolve over time. Regardless of what pattern is seen in the frontal region however, the pattern of the shift (morphology of the blue tracing) that is seen in the frontal region in each minute is very much the same as the pattern that is seen in that minute in the parieto-occipital region. This similarity in the morphology between the frontal and parieto-occipital shifts is also seen in the TMS responder (FIG. 5B). In the case of the two non-responders in FIG. 5C and FIG. 5D, there is very little similarity between the shift seen during each minute in the frontal region and those seen in the parieto-occipital region.

FIG. 6 is a series of graphs depicting the average individual alpha band for 18 different subjects. The mean peak alpha frequency between 7-14 Hz is identified for each subject prior to the start of treatment (black trace in each graph), which is designated as the center of each subject's individual 4 Hz-wide individual alpha band (dashed lines). Each 4 Hz-wide individual alpha band is examined for synchronization of shift between the frontal and parieto-occipital regions. In other words, each alpha band is examined for correlation in the blue lines for each minute between the frontal and parieto-occiptal regions.

FIG. 7 is a “spaghetti plot” depicting the synchronization in FIG. 6 over time. The left panel shows synchronization for remitters, the middle panels for responders, and the right panel for non-responders. The Y-axis shows the magnitude of the correlation and the X-axis shows the correlation for each of the minutes 1 through 6 following the end of a first TMS treatment. The correlation is the highest and the least variable for the remitters, somewhat high and somewhat more variable for the responders, and the lowest and most variable for the non-responders.

FIG. 8 depicts a set of heat maps between individual EEG electrode pairs within the frontal and parieto-occipital regions. Electrode pairs with high correlation are shown in red, while those with low or negative correlation are shown in blue. FIG. 8 shows a more complex pattern in which some areas show an increase and others show a decrease in synchronization between electrode pairs. These more detailed synchronization data recorded during the first treatment may be used to predict whether a patient is likely to respond to six weeks of TMS treatment.

FIG. 9 depicts the results of logistic regression analysis to predict response. The analysis shows that selecting a combination of four electrode pairs yields a prediction accuracy of up to 85%.

FIG. 10 is a graph depicting the proportion of patients responding to 10 Hz rTMS as a function of peak individual alpha frequency (IAF) (N=52). Deviation from 10 Hz shown on x-axis, with “0” indicating peak at 10 Hz, “−1” at 9 Hz, etc. Proportion of responders at each frequency on y-axis. Highest proportion of responders (red) clustered close to 10 Hz. Non-responders show broad distribution (black).

FIG. 11 is a graph depicting the probability of response to 10 Hz as a function of peak IAF. Subjects with IAF below 9.5 Hz (left) had 21%, 9.5 to 10.5 Hz (center) had 48%, and above 10.5 Hz had 31% response rate.

FIG. 12 is a schematic of the brain showing the frontoparietal control network (FCN), which includes the dorsolateral PFC (DLPFC), anterior inferior parietal lobule (aIPL), rostrolateral prefrontal cortex (rlPFC), region anterior to the supplementary motor area (preSMA), and inferior temporal gyms (ITG).

FIG. 13 is a series of graphs depicting examples of the pre-post spectral differences from the first rTMS session for a remitter (top row) and non-responder (bottom row) after 6 weeks of rTMS. The pre-post spectral difference is generated by subtracting each frequency bin in the pre-treatment EEG spectrum from the corresponding bin in the post-treatment spectrum. The resulting spectral difference graphs in a remitter show highly similar activity in the frontal (top left graph) and parietal (top right graph) regions, suggesting that the circuit bridging the two regions has been engaged in a similar manner. In the non-responder, the frontal and parietal regions are dissimilar (bottom left and right graphs), suggesting a lack of circuit engagement.

FIG. 14 is a graph depicting the mean±SD αDR value from baseline target interrogation in MDD subjects who went on to be remitters (red), responders (orange), and non-responders (gray) after 6 weeks of rTMS. αDR value was significantly higher in remitters and responders than non-responders, which was confirmed by the significant effect of the “group” variable using ANOVA (p=0.004).

FIG. 15 is a graph depicting the results of stepwise logistic regression for classification of remitters/responders vs. non-responders after 6 weeks of rTMS. αDR values from four electrode pairs distinguished the groups with 80% accuracy (p=0.0009).

FIG. 16 is a graph depicting the correlation between percent change in Inventory of Depressive Symptomatology (IDS) score after 6 weeks of rTMS (x-axis) and in αDR during target interrogation (y-axis) (r=0.44, p=0.007).

FIG. 17 is a graph depicting the percent difference in αDR value with target interrogation at IAF vs. 10 Hz. 15 subjects were interrogated at both frequencies, with 10 subjects (red bars on right) showing larger αDR value at IAF, and 5 (black bars on left) larger αDR value at 10 Hz. The percentage by which one magnitude exceeded the other is shown on the x-axis.

FIG. 18 is a flowchart detailing the study structure of a first phase study validating the measure of αDR for target engagement.

FIG. 19 is a flowchart detailing the study structure of a second phase study confirming target engagement and testing the superiority of individualized medicine approach based upon frequency selection.

FIG. 20 is a chart depicting the stratified randomization assignment to matched and mismatched treatment based upon pretreatment αDR interrogation.

FIG. 21 depicts an exemplary brain map provided by ANT Visor 2 neuronavigation for a posterior site.

FIG. 22 depicts an exemplary average e-field brain model for three subjects stimulated at DLPFC corrdinates. Areas shown in yellow-red have greater than 100% maximum current.

FIG. 23 depicts a computer simulation of the effects of different frequencies simulation on e-field. Stimulation frequencies between 4 and 30 Hz (x-axis) were evaluated for different stimulation amplitudes (y-axis, amplitude of current injected into neurons to simulate rTMS).

FIG. 24 depicts a flowchart of an exemplary target interrogation procedure to produce an αDR value.

FIG. 25 depicts the results of experiments demonstrating early improvement of anxiety symptoms. LO—left side treatment only, N=28. RL—right side treatment added in the second half of the treatment course. RE—Right side treatment added early, before treatment 15. Bars show the change in anxiety scores from pre-treatment baseline (black) to post-3 weeks of treatment (white). **—p<0.01; ***—p<0.01; n. s.—not significant.

FIG. 26 depicts an example of an αDR-Matrix. Seeds are located in the left and right frontal cortices, connecting to all other electrodes. Color code represents αDR intensity.

FIG. 27 depicts an αDR classification model. Left: Raw averages of features used for classification. 6/8 αDR values are lower for non-responders (x=1), than for responders (x=2). Asterisk indicate significant group averages of raw αDR values (no weighting). Right: AUC curves for 2 best elastic net models based on 8 selected features and yielding 67.7-70.9% accuracy.

FIG. 28 depicts αDR group averages for Left-Only and Right-Late treatment groups. Left: Left treatment only group; Right: Late right side treatment group. All αDR features are significantly higher for responders than for non-responders.

FIG. 29 depicts αDR values for a Right-Early group and a summary of AUC curves for all treatment groups.

FIG. 30 depicts an anatomical representation of selected features.

### DETAILED DESCRIPTION

The present invention provides methods for monitoring neuropsychiatric treatment of depression and other disorders. The methods monitor the progress of neuropsychiatric treatment by examining electrical oscillations in the brain as measured by quantitative electroencephalography (qEEG). The methods are useful in predicting and guiding the outcome of neuropsychiatric treatment.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Method of Monitoring Treatment of Neuropsychiatric Disorders

The present invention is in part based upon the measurement of quantitative electroencephalography (qEEG) to monitor the progress of neuropsychiatric treatments. In particular, the present invention describes a method for predicting outcomes of repetitive Transcranial Magnetic Stimulation (rTMS) for the treatment of Major Depressive Disorder (MDD). rTMS consists of electromagnetic pulses applied to a target brain region for the purpose of altering brain function. When the treatment is delivered five days per week for six weeks or longer, it leads to a substantial reduction in depressive symptoms. Patients commonly do not experience any relief of symptoms until several weeks post-treatment. rTMS treatment is costly and inconvenient, requiring a patient who may be disabled by MDD to travel to a treatment center on a daily basis. The present invention provides a method to determine early in the course of treatment whether rTMS is likely to be of benefit to a particular patient. If this method revealed that rTMS was unlikely to be effective, the rTMS administration can be modified or discontinued.

The mechanism of action (MOA) of rTMS in not completely understood. It is known that the initial effect of administering rTMS is the change patterns of electrical oscillations in the brain as measured by qEEG. rTMS may exert its therapeutic effects by altering these oscillations, resetting thalamocortical oscillatory circuits in the brain (Leuchter A F et al., Frontiers in human neuroscience 7 (2013): 37).

The method of the present invention assesses the activity of brain oscillatory circuits before, during, and after an initial treatment session. The method determines what patterns of oscillations are present in the brain immediately before the treatment and how the oscillations are affected by the treatment. By determining the nature of the oscillations at a pretreatment baseline as well as the change in oscillations with treatment, the method can be used to determine whether rTMS, as administered, is likely to lead to a remission of MDD.

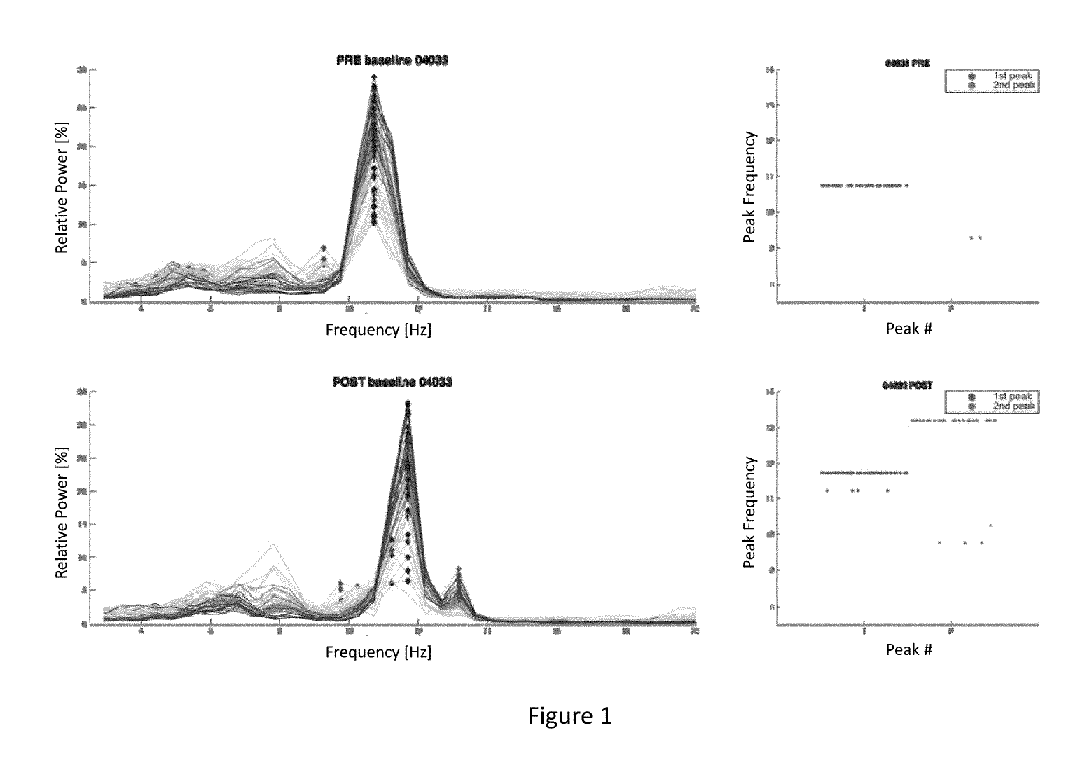

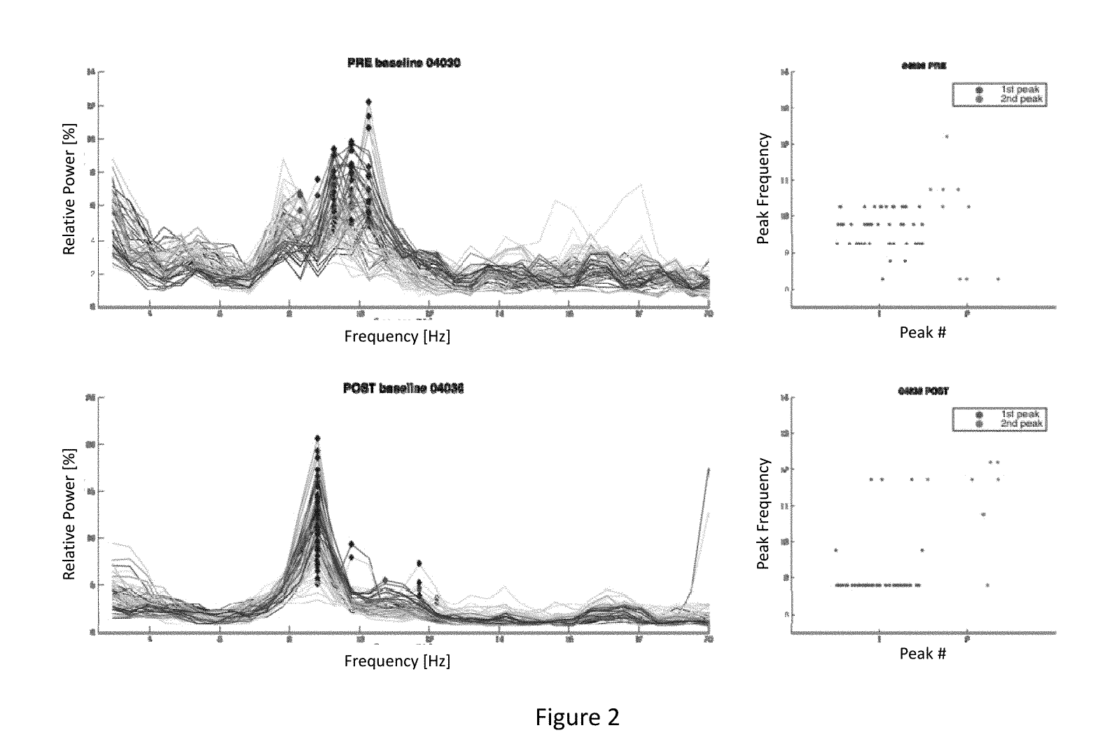

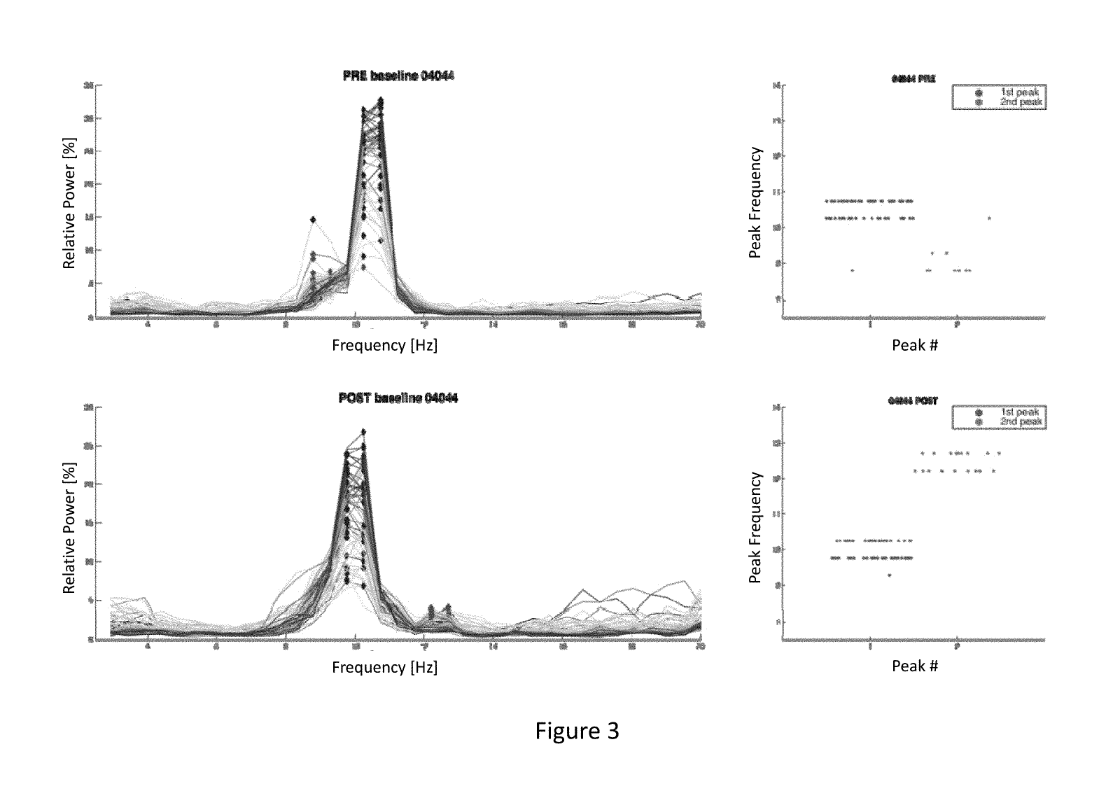

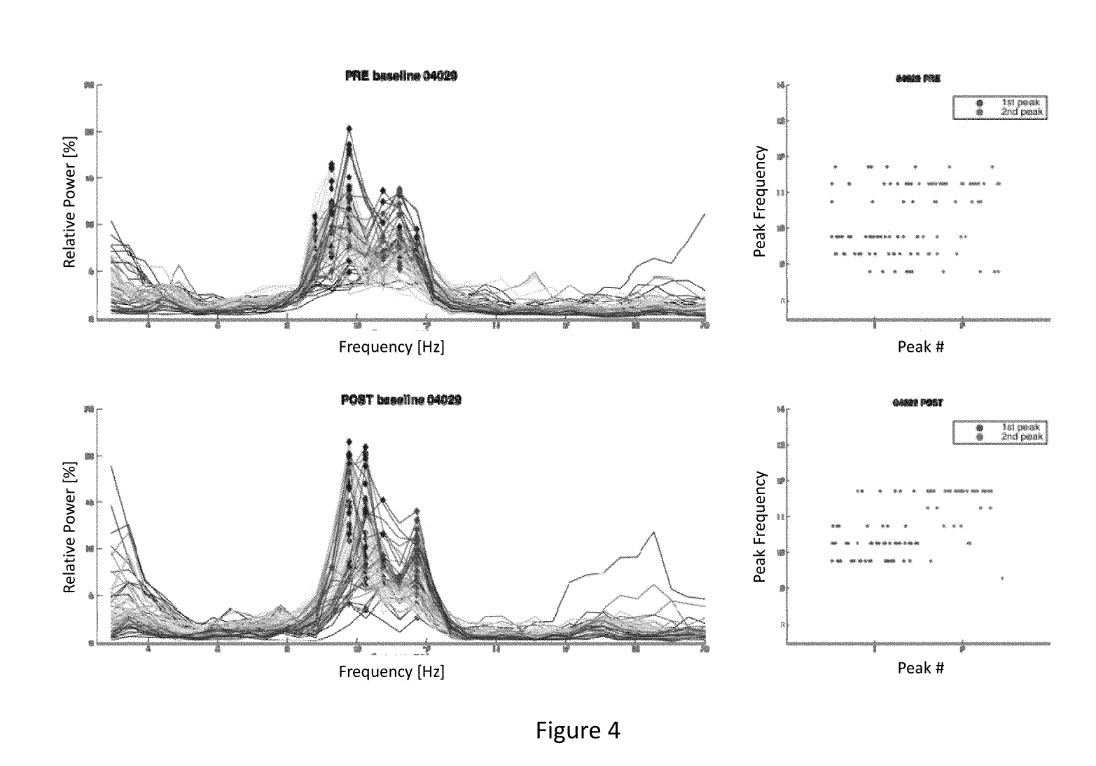

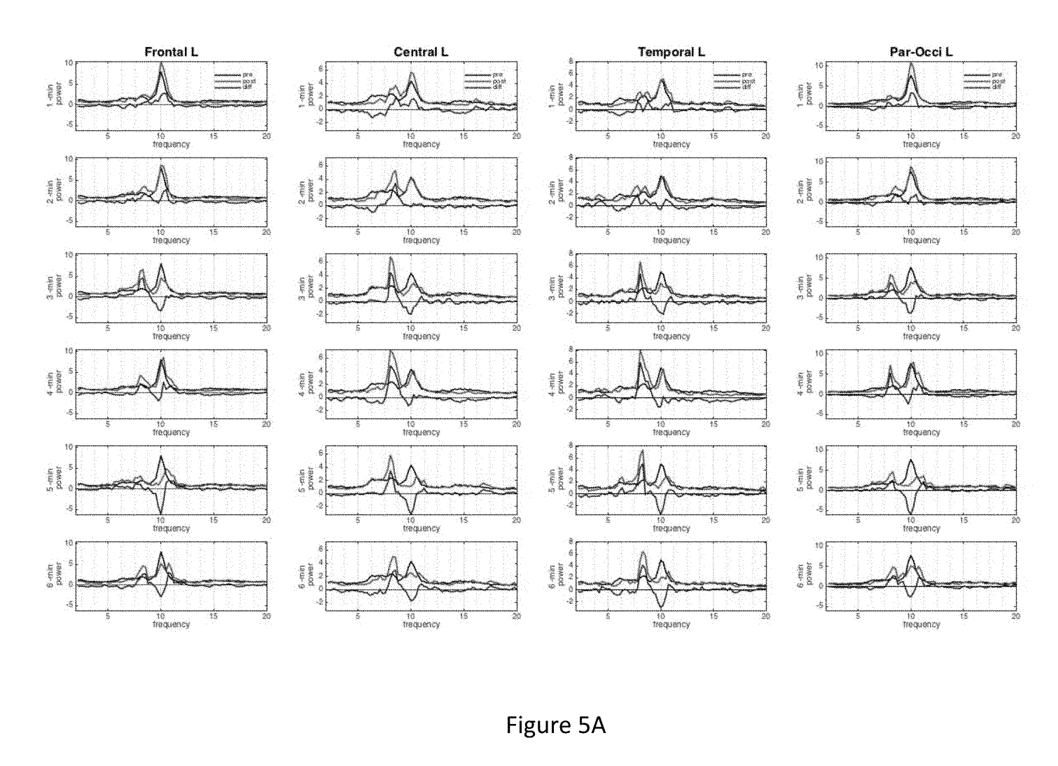

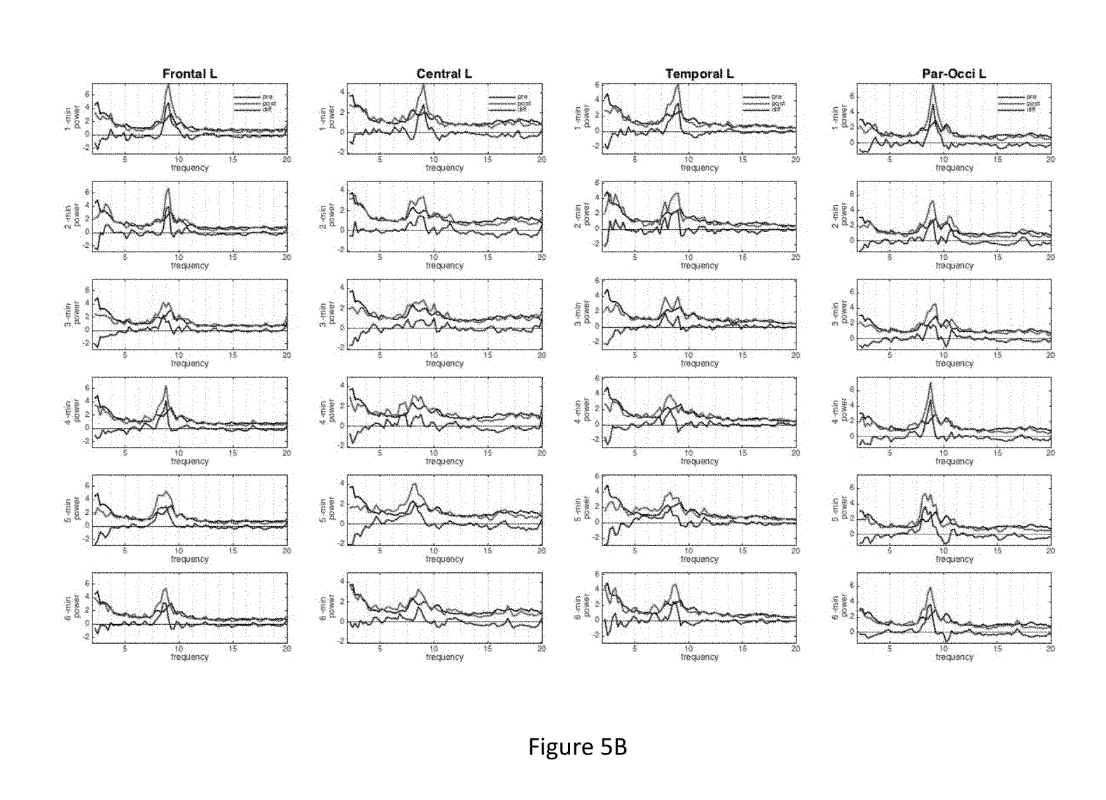

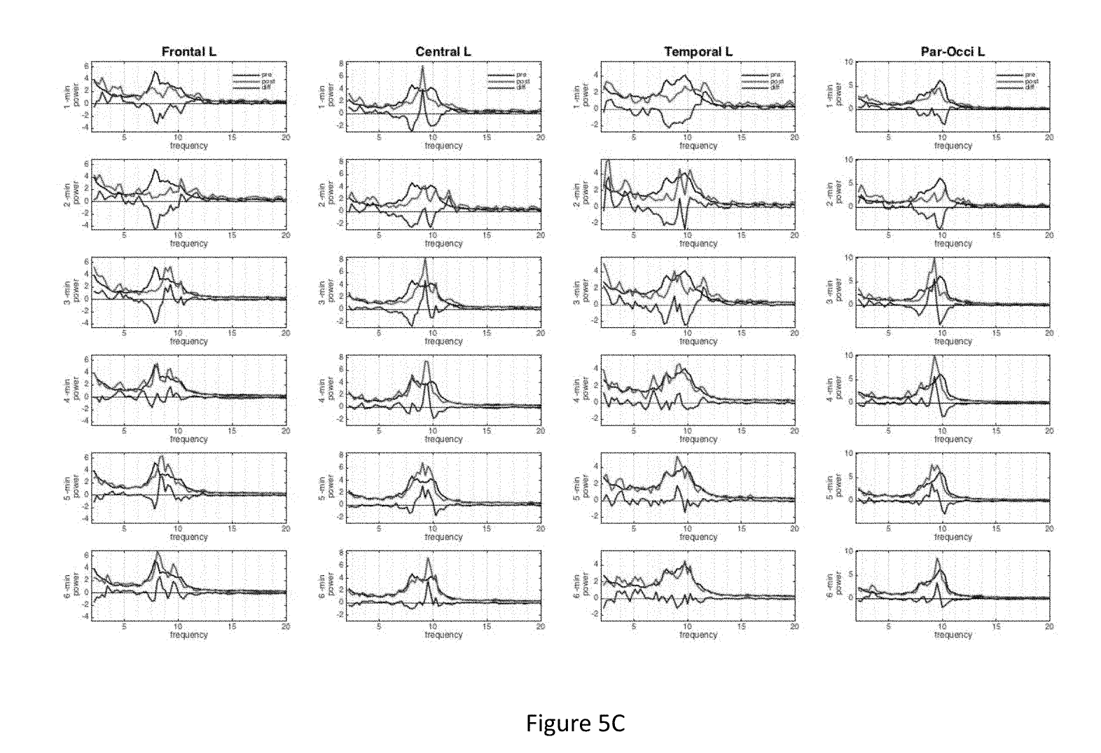

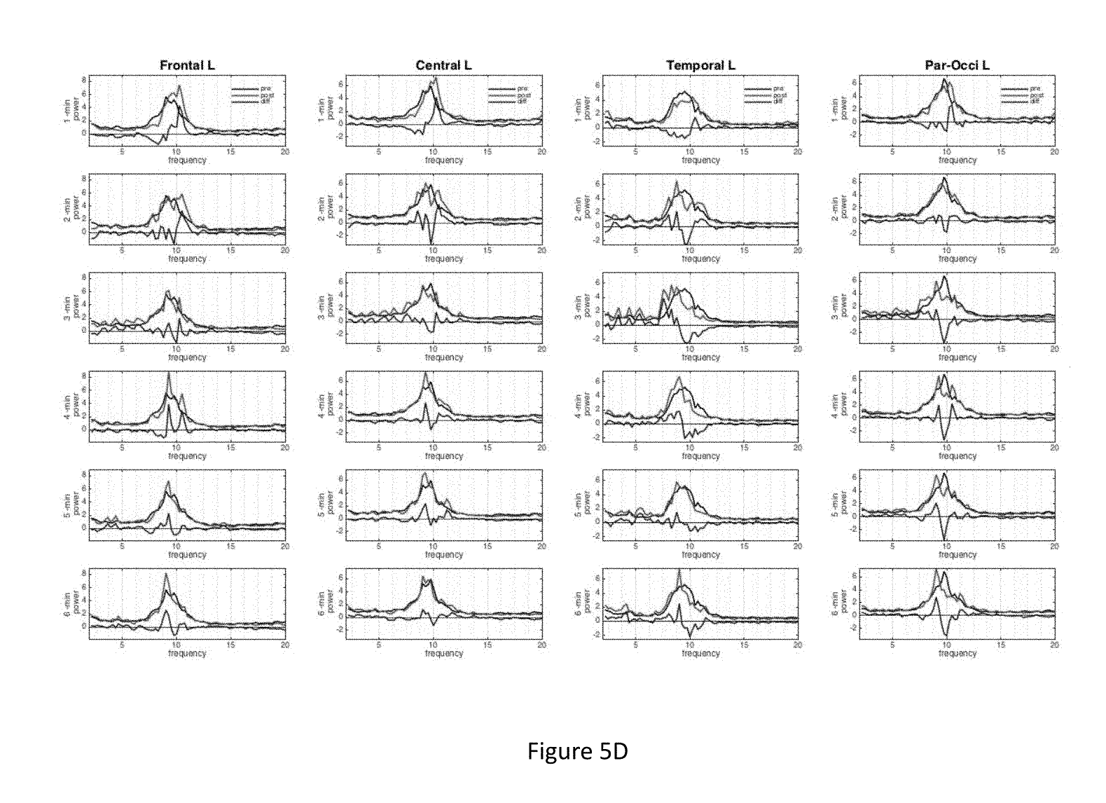

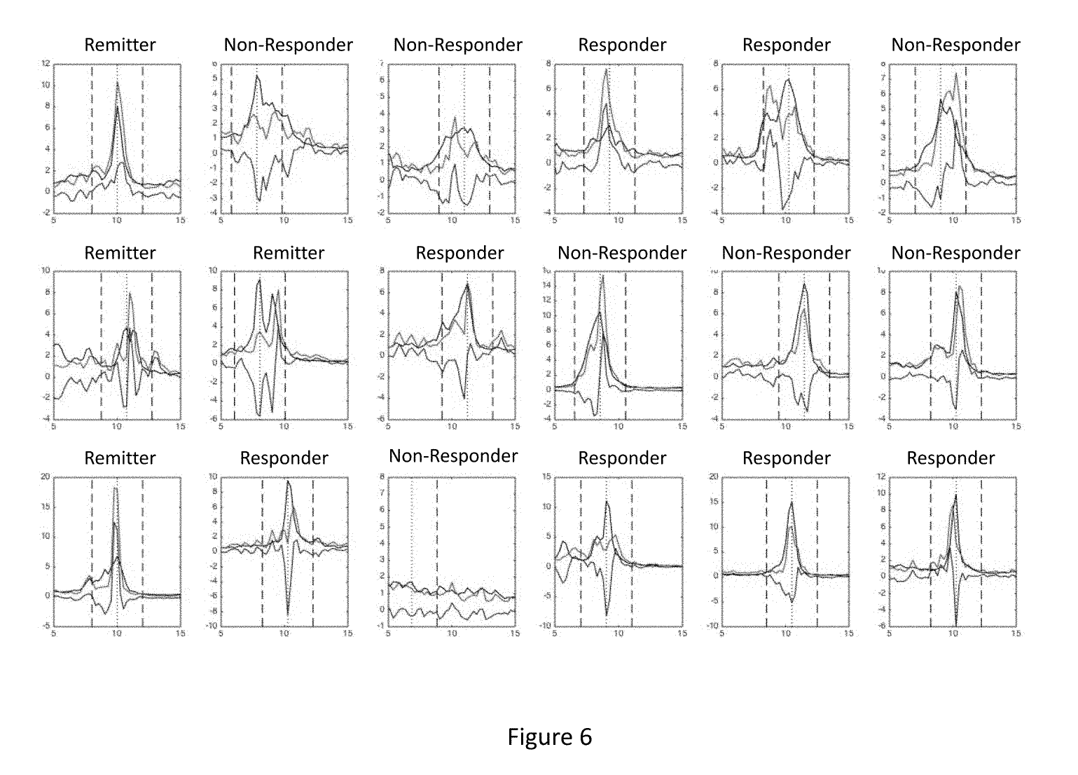

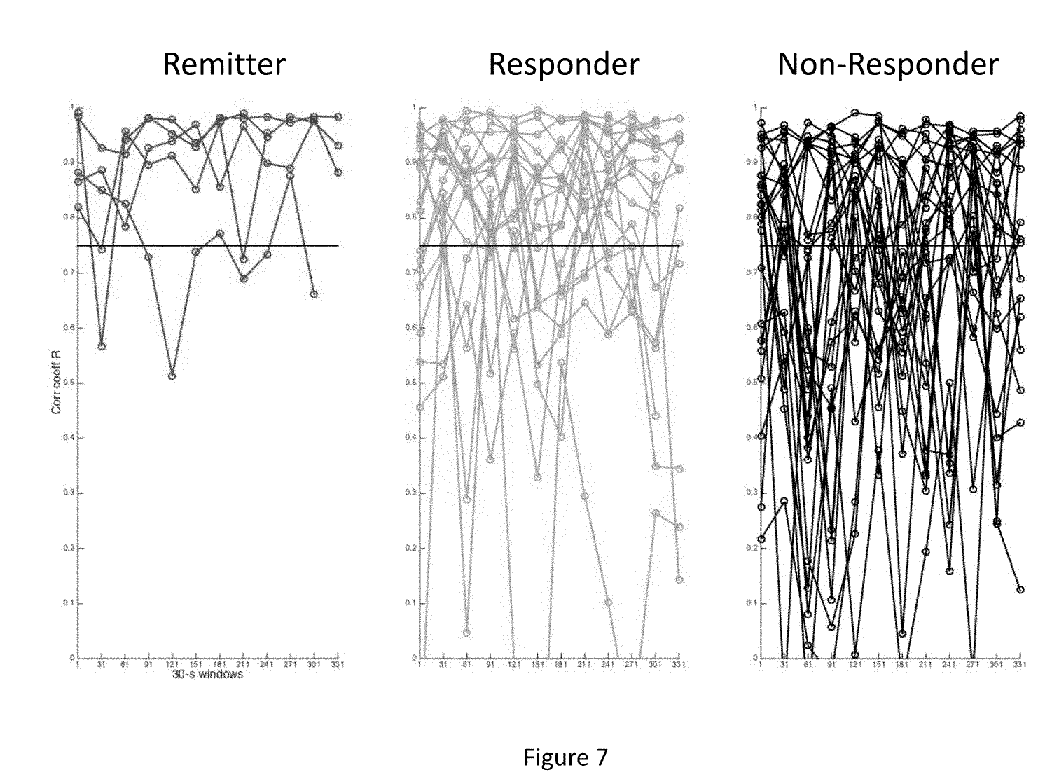

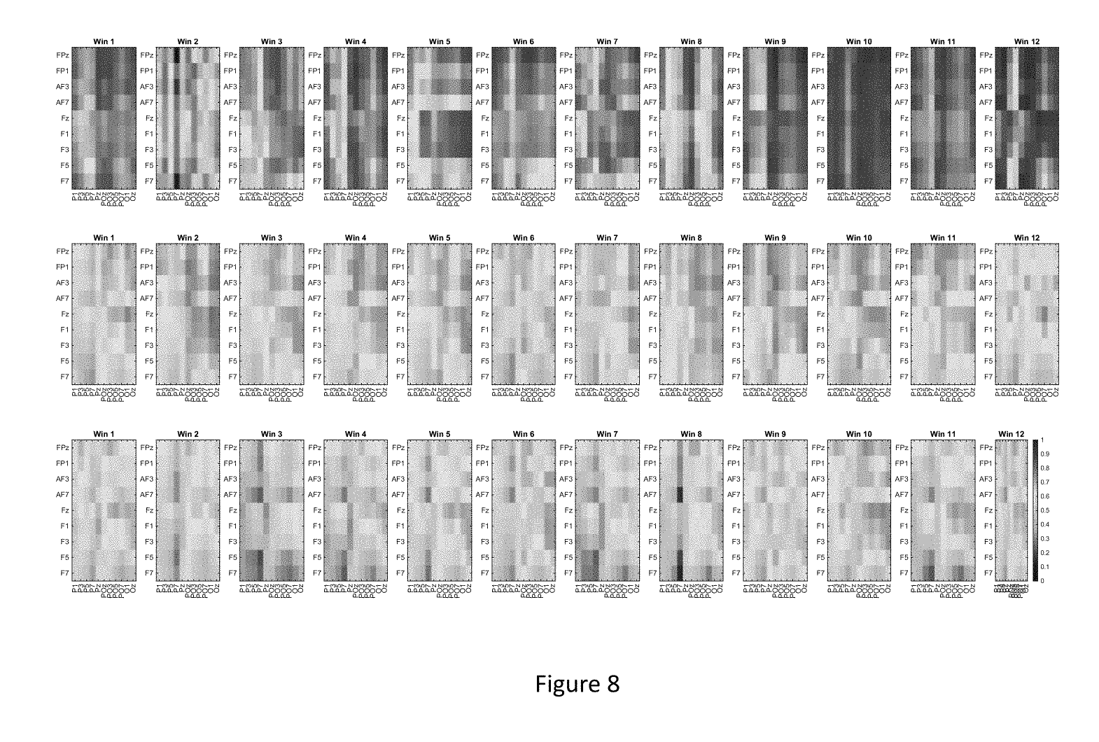

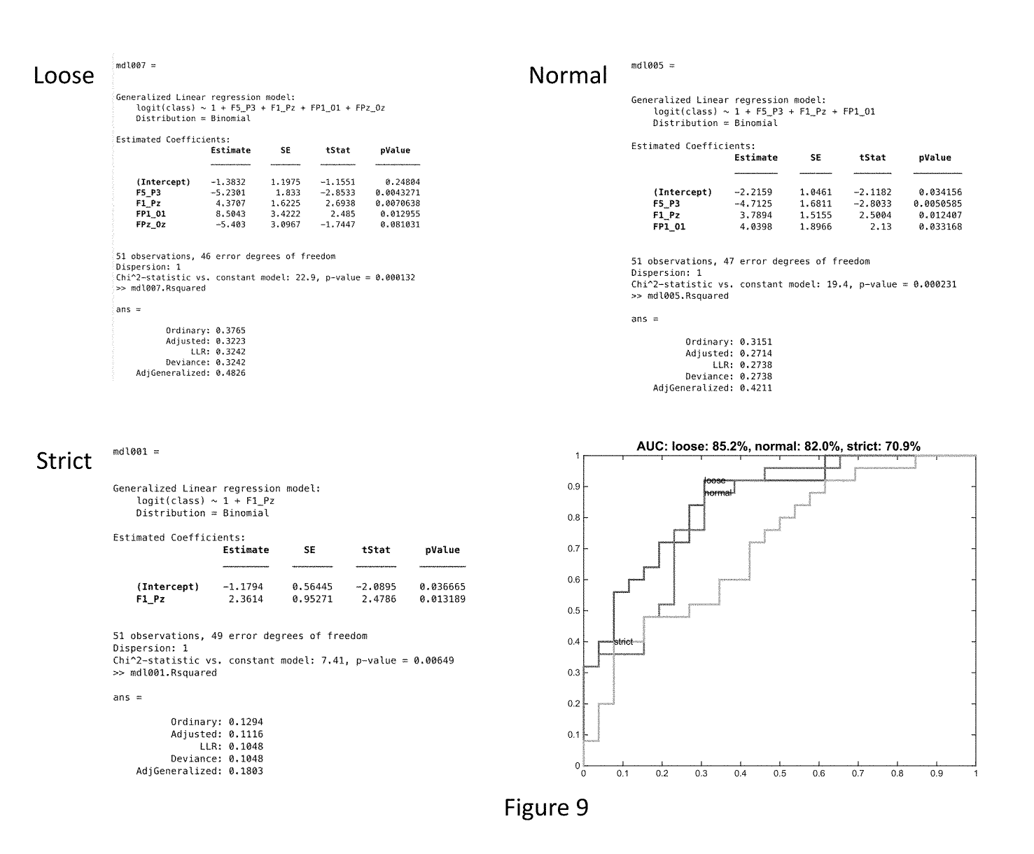

In some embodiments, the methods assess mean peak oscillatory patterns prior to, during, and following the first rTMS treatment session to assess the likely outcome of treatment. In some embodiments, a well-organized primary mean peak frequency prior to treatment may be an indicator of good prognosis (FIG. 1). An increase in the mean frequency of the primary peak and emergence of at least one higher frequency secondary peaks following the first treatment session demonstrate that rTMS treatment is highly likely to lead to a substantial reduction in depressive symptoms (FIG. 2, FIG. 3). Conversely, broad and less well-organized oscillatory primary mean frequency peaks prior to treatment that do not become better organized or increase in frequency, and are not accompanied by higher frequency secondary oscillatory peaks at the conclusion of the first treatment appear to be associated with poorer treatment outcome (FIG. 4).

In other embodiments, the methods assess the correlation in the difference between pre- and post-rTMS treatment mean peak frequency measured between at least two regions of the brain following the first treatment session to assess the likely outcome of treatment. A pre-rTMS treatment measurement can be taken immediately prior to the administration of an rTMS treatment session. A post-rTMS treatment measurement can be taken after a short delay, such as one minute. In some embodiments, a plurality of post-rTMS treatment measurements can be taken, such as six measurements spaced one minute apart.

The mean peak frequency can be measured in any of the brain's frequency bands. For example, in some embodiments the mean peak frequency can be in the delta band, between about 0.1 and 4 Hz. In some embodiments, the mean peak frequency can be in the theta band, between about 4 and 7 Hz. In some embodiments, the mean peak frequency can be in the alpha band, between about 7 and 13 Hz. In some embodiments, the mean peak frequency can be in the beta band, between about 13 and 30 Hz. In some embodiments, the mean peak frequency can be in the gamma band, between about 30 and 150 Hz.

The pre- and post-rTMS treatment mean peak frequencies can be measured in any of the brain's sites. Suitable brain sites include but are not limited to the left and right sides of: the frontal lobe, the central lobe, the temporal lobe, the parietal lobe, the occipital lobe, and combinations thereof. Further suitable brain sites include the left and right sides of: the various cortexes, such as the motor cortex, the premotor cortex, the prefrontal cortex, the somatosensory cortex, the posterior parietal cortex, the visual cortex, the auditory cortex, and the temporal cortex; the gyri, such as the frontal gyms, the postcentral gyms, the lateral occipital gyms, and the temporal gyms; the Brodmann Areas, such as Area 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 52; the cuneus; the precuneus; and combinations thereof.

In certain embodiments, the suitable brain sites can be selected or organized based on their inclusion in any one of the brain's networks. Contemplated brain networks include but are not limited to: the frontoparietal control network (FCN); the default mode network (DMN); the salience network (SN); the dorsal attention network (DAN); the ventral attention network (VAN); the basal ganglia network (BGN); the limbic network (LN); the somatomotor network (SMN); the visual network (VN); the frontoparietal network (FPN); the anterior insula network (AIN); the executive control network (ECN); the executive attention network (EAN); the medial visual network (MVN); the lateral visual network (LVN); the cerebellar network (CBLN); the auditory network (AN); the task positive network (TPN); the self-referential network (SRN); and the like.

In some embodiments, a high correlation in mean peak frequency between two brain sites may be an indicator of good prognosis. Conversely, a low correlation in mean peak frequency between two brain sites following the first rTMS treatment session may be an indicator of poor prognosis.

### Dynamic Response

The correlation in mean peak frequency between at least two brain regions can be quantified as a subject's dynamic response. The dynamic response is a biomarker that rates a subject's responsiveness to rTMS treatment and has a value of between −1 and 1, with a higher dynamic response value indicating better treatment response and a lower dynamic response indicating a worse treatment response. The dynamic response can be identified by the frequency of rTMS treatment, for example, the alpha dynamic response (αDR), the beta dynamic response (βDR), the gamma dynamic response (γDR), the delta dynamic response (ΔDR), and the theta dynamic response (θDR).

Dynamic response is calculated as the correlation of the shift between pre-rTMS treatment and post-rTMS treatment in at least two sites of the subject's brain. Generally, dynamic response is determined by the steps of: measuring a subject's pre-rTMS power spectrum at an EEG electrode of interest; calculating relative power from the subject's pre-rTMS power spectrum measurements at the EEG electrode of interest;

measuring a subject's post-rTMS power spectrum at the EEG electrode of interest;
calculating a relative power from the subject's post-rTMS power spectrum measurements at the EEG electrode of interest;
subtracting the post-rTMS power values from the pre-rTMS power values to obtain the shift in spectral power at the EEG electrode of interest; calculating correlation in spectral power shift between a pair of EEG electrodes positioned on at least two sites of a subject's brain; and averaging the correlations across the electrode locations.

Correlation can be assessed by calculating a correlation coefficient, such as Pearson's, Spearman's, or Kendall's. In some embodiments, correlation is determined using a Pearson's bivariate correlation coefficient, calculated using Eq. 1:

R=corr((S_{t1,F}−S_{t0,F}),(S_{t1,S}−S_{t0,S})) (Eq.1)

where S_{ti,Loc}=relative power of the spectrum measurements; tᵢ=t₀ for pre-rTMS; tᵢ=t₁ for post-rTMS; Loc=F for first brain site; and Loc=S for second brain site. In certain embodiments, dynamic response (DR) is calculated by averaging the correlation coefficients for a plurality of electrode pairs, such as in Eq. 2:

$$DR = \frac{{\sum }_{i = 1}^{n} ( {R}_{i} )}{n} \qquad ( Eq 2 )$$

where n=the number of channel pairs.

In certain embodiments, αDR is calculated using qEEG values recorded using electrode pairs from the FCN, in particular the F3, F5, Fp1, Fpz, and Pz electrode positions. The qEEG values can be recorded at a single pre- and post-rTMS treatment frequency, or over a range of pre- and post-rTMS treatment frequencies. In some embodiments, the frequency or the center of a range of frequencies is 10 Hz. In certain embodiments, αDR can be calculated by averaging the correlation coefficients for the four electrode pairs Fp1-Pz, Fpz-Pz, F3-Pz, and F5-Pz, in which case n=4. The calculated αDR value ranges from −1 to 1, with 1 representing a high degree of positively correlated shifts and strong TMS treatment response, and −1 representing negatively correlated shifts and weak TMS treatment response.

### Individual Alpha Frequency

The present invention also provides methods for adjusting rTMS treatment. As described elsewhere herein, rTMS generally involves the administration of a 10 Hz stimulation to the left dorsolateral prefrontal cortex (DLFPC). However, when the range of stimulation frequency is expanded, it is evident that the relative power of a subject's response differs between individuals. For certain subjects, the highest relative power is achieved at a frequency termed the individual alpha frequency (IAF).

In some embodiments, IAF is calculated using Welch's power spectral density estimate. A frequency power spectrum is generated by administering stimulations over a range of frequencies to a subject and recording the average power over the range. For example, in some embodiments, the stimulations are administered over a frequency range of between 2 and 20 Hz. In other embodiments, the stimulations are administered over a frequency range of between 5 and 15 Hz. Frequency resolution is dependent on sampling frequency and data length used to compute power spectra. In some embodiments, the obtained frequency resolution is 0.25 Hz, and the data length is based on a 4-second long artifact-free segment sampled at 1000 Hz.

In some embodiments, an IAF is determined as the single highest relative power peak within the stimulation range. In some embodiments, the IAF is determined as the single highest relative power peak within the stimulation range of 7 to 13 Hz. In some embodiments, the IAF is determined as the highest relative power peak within the 7 to 13 Hz stimulation range that surpasses a 95% confidence interval of the mean spectral power within the same range derived from a 2000-sample bootstrapped distribution. An IAF can be used as a single alpha peak frequency or as a baseline to establish an IAF band representing a range of therapeutically effective frequencies. For example, a 4 Hz IAF band represents a range of stimulation frequencies 2 Hz above and below the IAF.

As described above, certain subjects may be more responsive to TMS treatment administered at an IAF than at the standard 10 Hz stimulation. The difference in responsiveness can be quantified by determining the αDR value of a TMS treatment administered at an IAF and at the standard 10 Hz stimulation. A TMS treatment administered at an IAF would thereby output a higher αDR value than a TMS treatment administered at the standard 10 Hz stimulation.

It should be understood that while the IAF is discussed herein, the highest relative power can be any suitable frequency, such as the individual beta frequency (IBF), the individual gamma frequency (IGF), the individual delta frequency (IDF), and the individual theta frequency (ITF). Persons having skill in the art will understand that the frequency of stimulation and stimulation bands will be within the ranges appropriate for the respective frequency band.

In various embodiments, the methods of the invention can be used to aid decisions about whether and how to continue with rTMS treatment of neuropsychiatric disorders such as MDD at the end of an initial treatment session. Based upon these results, the treatment can be adjusted, such as by stimulating the brain in a different area, using a different pattern, or using a different pulse frequency in order to ameliorate symptoms. In other embodiments, the methods of the invention can also be used to guide treatment of other neuropsychiatric disorders including anxiety, PTSD, OCD, Parkinson's disease, and the like. The methods can also be applied to the use of other neuromodulation techniques, including but not limited to deep brain stimulation, transcranial direct current stimulation, and the like.

### Systems for Guiding Treatment of Neuropsychiatric Disorders

The present invention also provides software for guiding the treatment of neuropsychiatric disorders. The software combine one or more of the method described elsewhere herein to tune a subject's rTMS treatment: assessing mean peak oscillatory patterns prior to, during, and following a first rTMS treatment session, assessing the correlation between pre- and post-rTMS treatment mean peak alpha frequency measured in the frontal and parieto-occipital lobes following the first treatment session, determining a subject's dynamic response value for a particular TMS treatment frequency or range of frequencies, and determining a subject's individual frequency or individual frequency band.

In various embodiments, the present invention thereby includes software executing instructions and algorithms relating to the methods provided herein. Such software may be stored on a non-transitory computer-readable medium, wherein the software performs some or all of the steps of the present invention when executed on a processor.

Aspects of the invention relate to the algorithms executed in computer software. Though certain embodiments may be described as written in particular programming languages, or executed on particular operating systems or computing platforms, it is understood that the systems and methods of the present invention are not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

### Example 1: Four Subjects with MDD Treated with rTMS

Brain oscillation patterns of four subjects with MDD were monitored before and after treatment with rTMS. Subject 33 entered remission (i.e., had complete resolution of symptoms) at the conclusion of treatment. Subject 30 had a response to treatment (i.e., a 50% reduction in depressive symptoms). Subject 44 had improvement but less than a 50% reduction in symptoms. Subject 29 showed a worsening of symptoms with treatment. Prior to the first rTMS treatment, all subjects had a high density (64 channel) TMS compatible EEG electrode cap placed upon their head in order to allow recording of qEEG activity throughout the first recording session. FIG. 1 through FIG. 4 show the distribution peak mean frequency of oscillations in the 7.5 to 14 Hz frequency band. This band shows activity that is highly indicative of the activity of thalamocortical oscillators.

Subject 33 (remitted) shows a relatively narrow peak mean frequency pretreatment at slightly less than 11 Hz (top left graph of FIG. 1). This “primary” peak accounts for the great majority is energy within this frequency band, and there is no other “secondary” peak that accounts for a significant proportion of the energy in this band for this subject except for very few electrodes. The presence and frequency of this primary peak is indicated by the row of blue dots in the top right panel of FIG. 1, and the secondary peak is indicated by the red dots, with each dot representing individual EEG recording electrodes (some dots may be superimposed on one another). After treatment, the mean frequency of this primary peak has increased to nearly 12 Hz and has narrowed, indicating a tighter distribution around this higher mean frequency. Additionally, a secondary faster peak has developed at nearly 13 Hz (bottom left graph of FIG. 1). The presence and frequency of these primary and secondary peaks following treatment also are shown in the bottom right graph of FIG. 1. These results show the pattern of oscillations that appears to be associated with the best treatment outcome: a single well-organized peak of oscillations prior to the first treatment that increases in frequency and narrows in distribution with treatment and is accompanied by the emergence of a secondary peak of even higher frequency.

Subject 30 (responded) shows a different pattern prior to treatment, with no single well organized peak in the frequency range (top left graph of FIG. 2). This disorganized oscillatory pattern of primary and secondary peaks is indicated by multiple rows of dots in the top right graph of FIG. 2. Following the first treatment session, the pattern of oscillations becomes much better organized with the emergence of a relatively narrow primary peak, albeit at a lower frequency within the range. There are also secondary peaks higher in the frequency range that emerge for a few electrodes (bottom graphs of FIG. 2). These results show a pattern of oscillations that appears to be associated with response, but an incomplete resolution of depressive symptoms: a disorganized pattern of oscillations prior to treatment that becomes better organized with treatment, but with a lower mean peak primary oscillatory frequency and relatively fewer electrodes showing a higher peak mean frequency.

Subject 44 (improved) demonstrates a pattern that is associated with improvement but neither remission nor response. Prior to treatment, there is a broad “double” primary peak frequency between 10 and 11 Hz and several electrodes that show secondary peaks at around 9 Hz (top row of graphs of FIG. 3). Following treatment, the primary peak maintains this broad double peak pattern, does not become better organized, and slows in mean frequency to between 9.5 and 10.5 Hz. There is the emergence, however, of faster secondary peaks for many electrodes at the high end of the frequency range (bottom row of graphs of FIG. 3). These results show a pattern associated with improvement in which the primary peak is relatively well-organized prior to treatment but slows and does not become better organized with treatment, and is accompanied by the emergence of higher frequency secondary peaks that are also not well-organized.

Subject 29 (worsened) shows a pattern that is associated with exacerbation of depressive symptoms with rTMS (FIG. 4). This subject shows many poorly organized primary and secondary mean frequency peaks prior to treatment (top row of graphs of FIG. 4). After treatment, the multiple primary and secondary peaks remain poorly organized although they increase slightly in mean frequency (bottom row of graphs of FIG. 4).

### Example 2: Dynamic Response of the Frontoparietal Control Network (FCN) to Stimulation in the α Frequency Range

Repetitive Transcranial Magnetic Stimulation (rTMS) for treatment of Major Depressive Disorder (MDD) commonly is administered to left dorsolateral prefrontal cortex (DLPFC) at a frequency of 10 Hz (McClintock S M et al., The Journal of clinical psychiatry, 2017). The immediate effect of rTMS is entrainment of oscillations in underlying cortex to the frequency of stimulation (Thut G et al., Current biology, 2011, 21(14):1176-1185). This change in oscillations rapidly spread through brain networks to related brain regions (Hanlon C A et al., PLoS One, 2013, 8(7):e67917). Entrainment of oscillations is expected to “reset” thalamocortical oscillators and may be related to the therapeutic mechanism of rTMS (Leuchter A F et al., Frontiers in human neuroscience, 2013, 7; Leuchter A F et al., Annals of the New York Academy of Sciences, 2015, 1344(1):78-91). Frequency of stimulation therefore constitutes an important target to consider for enhancing the efficacy of rTMS treatment.

10 Hz is the center of the alpha (α) frequency band. α oscillations represent a thalamocortical rhythm (Bollimunta A et al., Journal of Neuroscience, 2011, 31(13):4935-4943), and therefore optimizing the targeted frequency band may help recruit thalamocortical oscillators more effectively and result in a stronger therapeutic effect. The research presented herein addresses a key unanswered question: is 10 Hz best stimulation target frequency in the α band? The range and peak frequency of α oscillations vary across individuals (Haegens S et al., Neuroimage, 2014, 92:46-55), with the intrinsic resonant frequency of a network best defined by its peak frequency (Touzel M P et al., PLoS computational biology, 2015, 11(12):e1004636). These findings demonstrate that an individualized medicine approach in which the rTMS stimulation frequency is tuned to each subject's peak individual a frequency (IAF) may enhance rTMS efficacy.

The following study investigates an individualized medicine approach to rTMS in which the frequency of stimulation will be “tuned” to an α frequency that is most likely to provide therapeutic benefit. The study validates a novel rTMS target: dynamic response of the frontoparietal control network (FCN) to stimulation in the α frequency range. FCN is focused on because: 1) the network is dysregulated in MDD, 2) DLPFC is a critical node of FCN; 3) FCN integrates multiple resting state networks (RSNs); and 4) FCN activity is coordinated by a oscillations (7-13 Hz) that vary in frequency across individuals. The α dynamic response (αDR) target is a quantitative electroencephalography (qEEG) measure that assesses response of FCN during an α-frequency rTMS “challenge” (target interrogation) delivered to DLPFC. In 52 MDD patients monitored during their first exposure to 10 Hz rTMS, it was found that the αDR measure predicted remission/response to a six-week course of daily rTMS therapy with 80% accuracy, and the magnitude of αDR was strongly correlated with degree of clinical improvement. Separately, it was found that target interrogation using stimulation at peak IAF generated a higher magnitude αDR signal than 10 Hz stimulation in 66% of subjects. Taken as a whole, these data support the approach that the αDR target can be used to interrogate FCN at different a band stimulation frequencies, and to select an individually-targeted frequency to yield a therapeutic response.

The study tests the highly innovative approach of interrogating a single neuroanatomic target (left DLPFC) with different frequencies of stimulation in the α frequency band as broadly defined in previous IAF stimulation studies (7-13 Hz) (Klimesch W et al., European Journal of Neuroscience, 2013, 17(5):1129-1133). This approach builds on work indicating that neuronal circuits have preferred resonant frequencies (Hutcheon B et al., Trends in neurosciences, 2000, 23(5):216-222). Low intensity current stimulation (tACS) of a circuit with preferred frequency in the α range at its IAF has been shown to upregulate a oscillations and may enhance spike-timing dependent plasticity (STDP) (Zaehle T et al., PloS one, 2010, 5(11):e13766). With regard to rTMS, recent work has established that different frequencies of stimulation applied to a single neuroanatomic target have distinct effects on engagement of resting state networks (RSNs) during rTMS stimulation. Adjusting the frequency of stimulation affects which nodes within a single RSN are engaged (Salinas F S et al., Brain stimulation, 2016, 9(3):406-414; Eldaief M C et al., Proceedings of the National Academy of Sciences, 2011, 108(52):21229-21234), the extent to which rTMS stimulation changes local vs. distant network modules (Davis S W et al., Human Brain Mapping, 2017), and can selectively alter the engagement between an RSN and an affiliated brain region (Vanderhasselt Mass. et al., Experimental brain research, 2006, 169(2):279-282). Specifically with regard to the frontoparietal control network (FCN), different stimulation frequencies applied to a parietal node can change FCN interactions with default mode network (DMN) from excitatory to inhibitory (Chen A C et al., Proceedings of the National Academy of Sciences, 2013, 110(49):19944-19949). Taken as a whole, this evidence supports the approach of interrogating the effects of different frequencies of stimulation at a single neuroanatomic target.

10 Hz stimulation of left DLPFC is the most common form of rTMS, and formed the basis for FDA approval for treatment of MDD (McClintock S M et al., The Journal of clinical psychiatry, 2017). 10 Hz is the middle of the α band, and the present study refines a band stimulation using an individualized medicine approach, in particular two specific frequencies within this band (peak IAF vs. 10 Hz). Other useful frequencies within the α band, as well as other frequencies of stimulation that have been reported to be efficacious for treatment of MDD (i.e., 1, 5, 20 Hz, and theta burst). The initial portions of the study validates the concept and target of dynamic response (DR) of a brain circuit to α stimulation (αDR).

The initial focus on the α band is grounded in the central role of α oscillations in organizing the activity of brain networks (Leuchter A F et al., Frontiers in human neuroscience, 2013, 7). α oscillatory activity binds together the activity of brain regions within many resting state networks (RSNs) and routes flow of information in the cortex (Wang L et al., Neuron, 2012, 76(5):1010-1020; Popov T et al., Journal of Neuroscience, 2017, 37(15):4117-4127). Furthermore, fluctuations in the local fMRI BOLD signal are strongly related to spontaneous α oscillations which largely explain evoked fMRI response variance (Sadaghiani S et al., Journal of Neuroscience, 2010, 30(30):10243-10250; Becker R et al., Journal of Neuroscience, 2011, 31(30):11016-11027). α band oscillatory activity over brain areas topographically related to the DMN also show high correlations with spontaneous self-referential thoughts and other DMN related functions both in the resting state and during a task (Knyazev G G et al., Brain research, 2011, 1402:67-79). Evidence indicates that FCN connectivity is mediated by a oscillations in the 7-13 Hz frequency band (Hacker C D et al., NeuroImage, 2017, 149:446-457; Wallis G et al., Journal of Cognitive Neuroscience, 2015; Hsueh J J et al., Human brain mapping, 2016, 37(7):2662-2675). Finally, α band oscillatory activity is significantly disturbed in MDD (Leuchter A F et al., PLoS One, 2012, 7(2):e32508), and it has been reported that shifts towards increased α frequency activity early in the course of antidepressant treatment predict medication (but not placebo) response/remission (Leuchter A F et al., Journal of psychiatric research, 2017, 84:174-183). a frequencies therefore constitute a logical target frequency for this individualized medicine rTMS investigation.

As noted above, IAF may have greater efficacy at engaging brain networks that oscillate in the α frequency range by taking advantage of their resonant properties (Hutcheon B et al., Trends in neurosciences, 2000, 23(5):216-222; Zaehle T et al., PloS one, 2010, 5(11):e13766). While mean a frequency (i.e., the average frequency of oscillations in the α band) can vary over time within an individual, peak IAF (i.e., the single largest oscillatory peak in the α band) is a highly stable neurophysiological trait marker in healthy younger and older adults, with high reliability upon multiple measurements over up to six months (Grandy T H et al., Psychophysiology, 2013, 50(6):570-582). In support of this application, EEG data from an earlier treatment trial was reanalyzed for subthreshold TMS stimulation at the IAF (sTMS) in MDD (Leuchter A F et al., Brain stimulation, 2015, 8(4):787-794). EEG data from 170 subjects who had an average of 8.2 EEG recordings each over the span of six weeks of treatment was examined to determine the stability and reliability of peak IAF measurements over time in MDD. While the mean a frequency has a standard deviation of 0.8 Hz (consistent with values reported in the literature), peak IAF such as proposed in this study had a standard deviation of only 0.32 Hz. These data demonstrate that IAF as operationalized here will have favorable stability characteristics.

Several lines of evidence indicate that TMS stimulation at IAF may have behavioral effects that are superior to uniformly applied 10 Hz stimulation. One study that reported that stimulation at IAF+1 Hz was associated with greater enhancement of cognitive task performance than with stimulation at slower or faster frequencies (Klimesch W et al., European Journal of Neuroscience, 2013, 17(5):1129-1133). It is expected that treatment based upon stimulation at a subject's IAF would be more effective than 10 Hz stimulation at entraining oscillatory activity at the site of stimulation (Leuchter A F et al., Frontiers in human neuroscience, 2013, 7), although no previous study has examined the therapeutic efficacy of rTMS performed at IAF at or above a patient's motor threshold. In a proof-of-concept study, subthreshold TMS stimulation at the IAF (sTMS) was reported to be an effective treatment for MDD (Leuchter A F et al., Brain stimulation, 2015, 8(4):787-794). It was also reported that low intensity a-band stimulation delivered at a frequency that deviated from IAF yielded poorer outcomes (based on per protocol and not ITT analysis) (Leuchter A F et al., Brain stimulation, 2015, 8(4):787-794; Leuchter A F et al., Brain Stimulation: Basic, Translational, and Clinical Research in Neuromodulation, 2017, 10(2):492). In a small pilot study of 18 patients with MDD, rTMS treatment at IAF+1 was not superior (and may have been inferior) to 10 Hz treatment (Arns M et al., Brain Stimulation: Basic, Translational, and Clinical Research in Neuromodulation, 2010, 3(2):124-126), but no patients in that study were treated at their actual IAF. A study of 90 subjects with MDD did report a relationship between peak IAF recorded from frontal electrode sites and likelihood of response to rTMS (Arns M et al., Brain stimulation, 2012, 5(4):569-576). One report from the OPT-TMS trial failed to find a relationship between peak α frequency and outcome (Widge A S et al., Brain stimulation, 2013, 6(6):929-931). This study, however, appears to have calculated peak α frequency from the mean spectrum, not the true primary α peak as examined here. It also is important to note that, unlike OPT-TMS, the present study records EEG using electrodes pairs from the frontal region (left DLPFC), which were found to differ from other regions (data not presented). Furthermore, as the OPT-TMS authors noted, technical difficulties with EEG data collection in OPT-TMS limited the generalizability of their results.

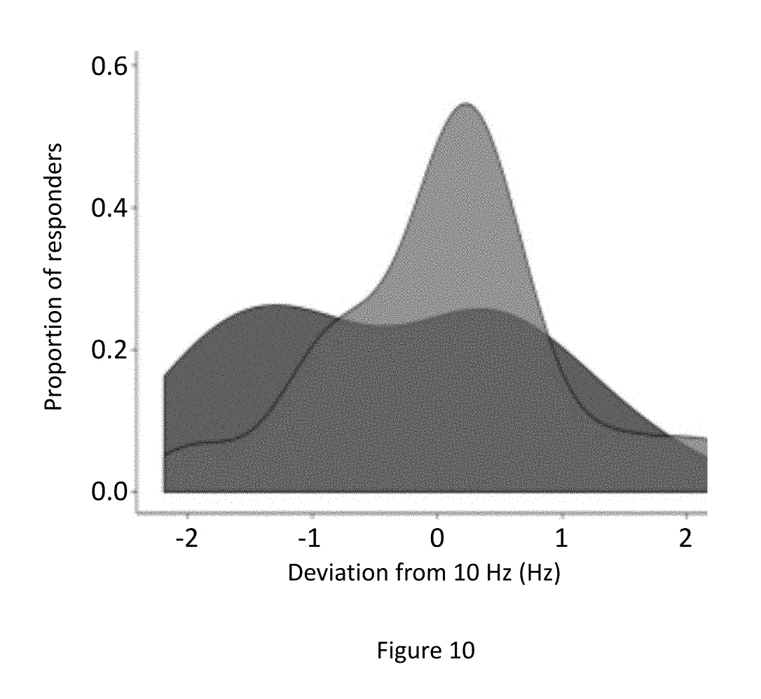

In support of this study, the clinical response to rTMS was examined in relation to each subject's IAF in 52 patients treated for six weeks with 10 Hz stimulation to left DLPFC (FIG. 10). The highest proportion of responders was seen among subjects whose peak IAF was at 10 Hz (in red). There was a notable drop off in proportion of responders with increasing distance from the 10 Hz stimulation (on x-axis). Non-responders (in black) showed no relation to stimulation frequency.

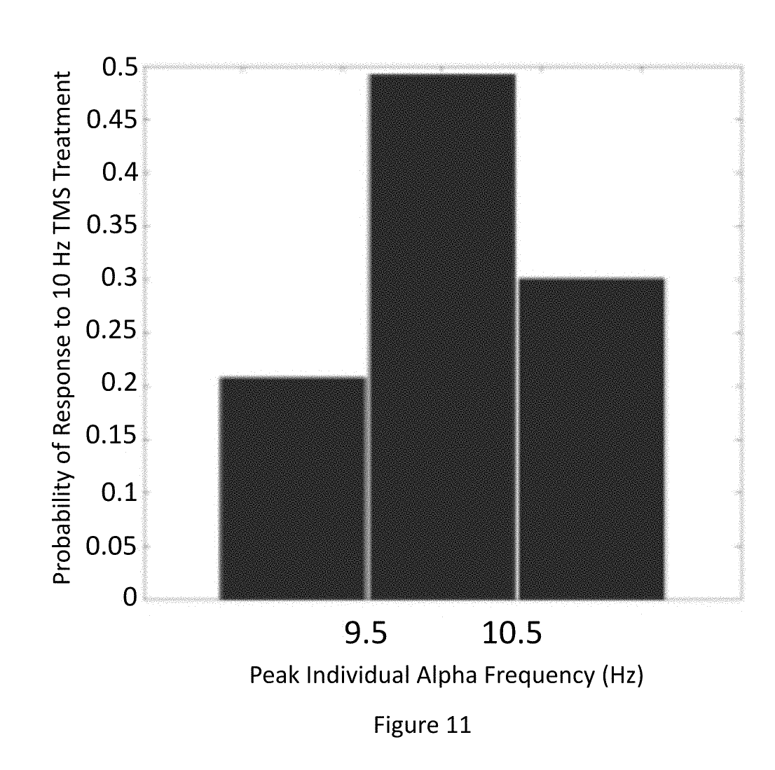

The likelihood of a patient meeting response criteria as a function of their peak IAF frequency was also examined for these same patients (FIG. 11). The overall sample had a response rate of 37%, which was distributed unevenly across the frequency spectrum. Subjects whose peak IAF was between 9.5-10.5 Hz had a notably higher response rate (48%), while those below 9.5 Hz had a lower rate (21%) as did those above 10.5 Hz (31%). On average, those outside of a 1 Hz band surrounding 10 Hz had approximately half the likelihood of responding to 10 Hz rTMS as those within the 1 Hz band. These data, along with the scientific literature reviewed above, support the investigation of rTMS pulse rate frequencies within subjects' IAF range as a personalized medicine strategy for treatment of MDD.

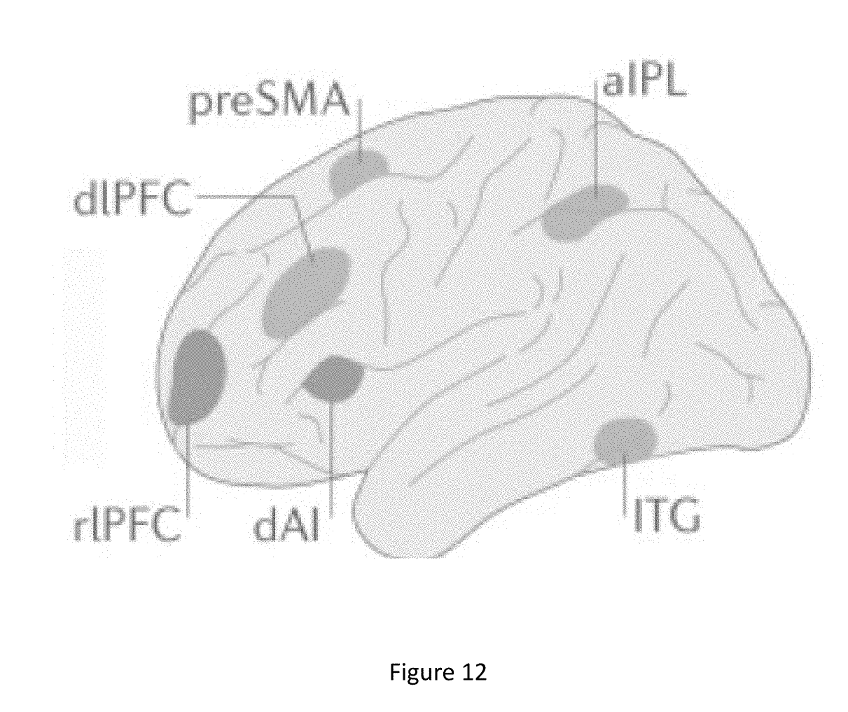

FCN was chosen as the focus because: 1) the FCN is dysregulated in MDD, with the degree of dysfunction related to severity of depressive symptoms (Liston C et al., Biological psychiatry, 2014, 76(7):517-526; Kaiser R H et al., JAMA psychiatry, 2015, 72(6):603-611; Hyett M P et al., JAMA psychiatry, 2015, 72(4):350-358; Seeley W W et al., Journal of Neuroscience, 2007, 27(9):2349-2356), 2) DLPFC is a critical hub of the network (Christoff K et al., Nature Reviews Neuroscience, 2016, 17(11):718-731), 3) FCN integrates the function of multiple RSNs (Kuhn S et al., Schizophrenia bulletin, 2011, 39(2):358-365; Buchanan A et al., Journal of psychiatric research, 2014, 59:38-44; Sylvester C M et al., Trends in neurosciences, 2012, 35(9):527-535), and, 4) it is known that activity in FCN is coordinated by α oscillations (7-13 Hz) that vary in frequency across individuals (Hacker C D et al., NeuroImage, 2017, 149:446-457; Wallis G et al., Journal of Cognitive Neuroscience, 2015; Hsueh J J et al., Human brain mapping, 2016, 37(7):2662-2675). Finally, the major brain regions within the FCN all are located on the cerebral convexities under EEG recording electrodes (Christoff K et al., Nature Reviews Neuroscience, 2016, 17(11):718-731), making them amenable to direct monitoring by qEEG (FIG. 12).

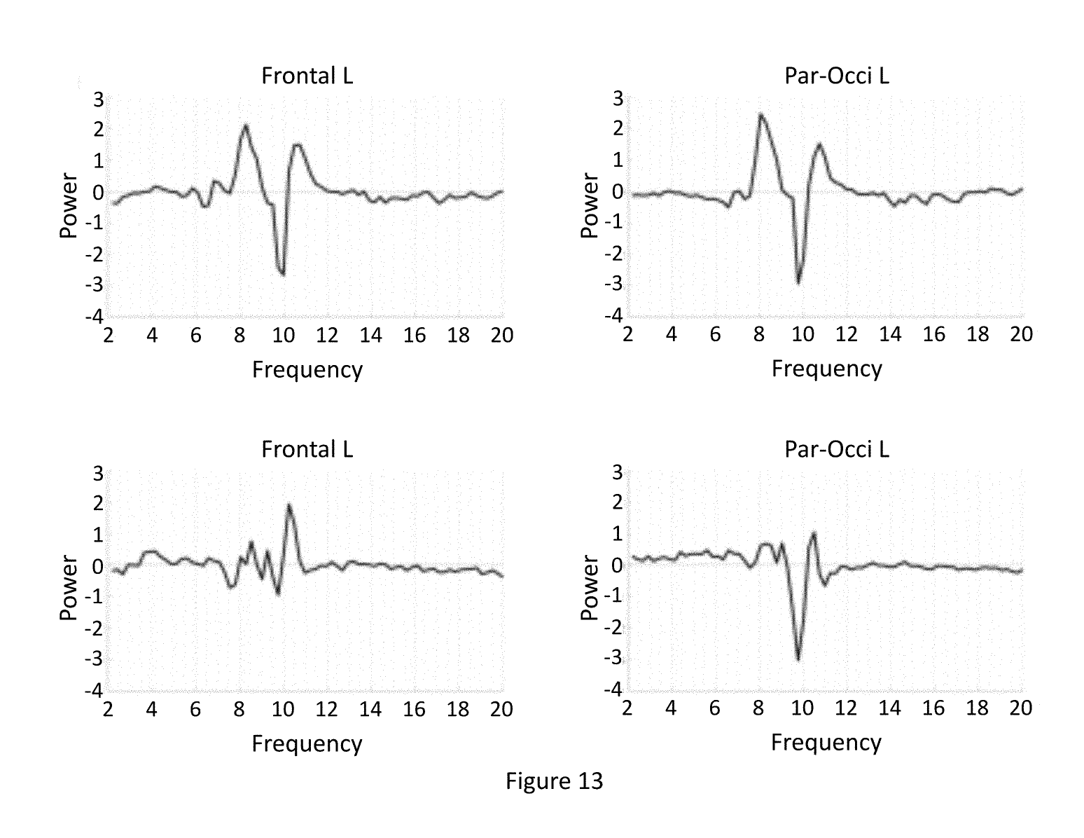

αDR is a novel biomarker that is analogous to other EEG measures of circuit engagement. Based upon an rTMS “target interrogation” session, αDR estimates shared rTMS-induced spectral perturbations in each subject's IAF band between electrodes in the prefrontal and parietal regions of the FCN. This measure was developed based upon the empiric observation that in qEEG recorded in the first rTMS treatment session, the change in the power spectrum in an individually defined a band was very similar between frontal and parietal electrode sites in subjects who went on to remission after 30 sessions (FIG. 13, top row), but not in subjects who failed to respond to treatment (FIG. 13, bottom row). These spectral differences were not detectable with traditional EEG connectivity measures such as coherence (data not presented). αDR was therefore developed as a measure that is based on temporal-pattern similarity analysis, which has been used by other investigators to demonstrate shared spectral activity between brain regions that are encoding common information (Staudigl T et al., Journal of Neuroscience, 2015, 35(13):5373-5384). Similar methods also have been employed not only to study encoding of information, but also to examine information processing in the context of the effects of TMS stimulation (Hanslmayr S et al., Journal of Neuroscience, 2012, 32(42):14742-14751).

The αDR-measure is calculated as the correlation of spectral changes from pre- to post rTMS treatment between frontal and parietal sites for each subject's IAF band (IAF±2 Hz). Specifically, electrodes overlying the neuroanatomic regions of the FCN (F3, F5, Fp1, Fpz, Pz) were selected to calculate αDR (Koessler L et al., Neuroimage, 2009, 46(1):64-72; Christoff K et al., Nature Reviews Neuroscience, 2016, 17(11):718-731).

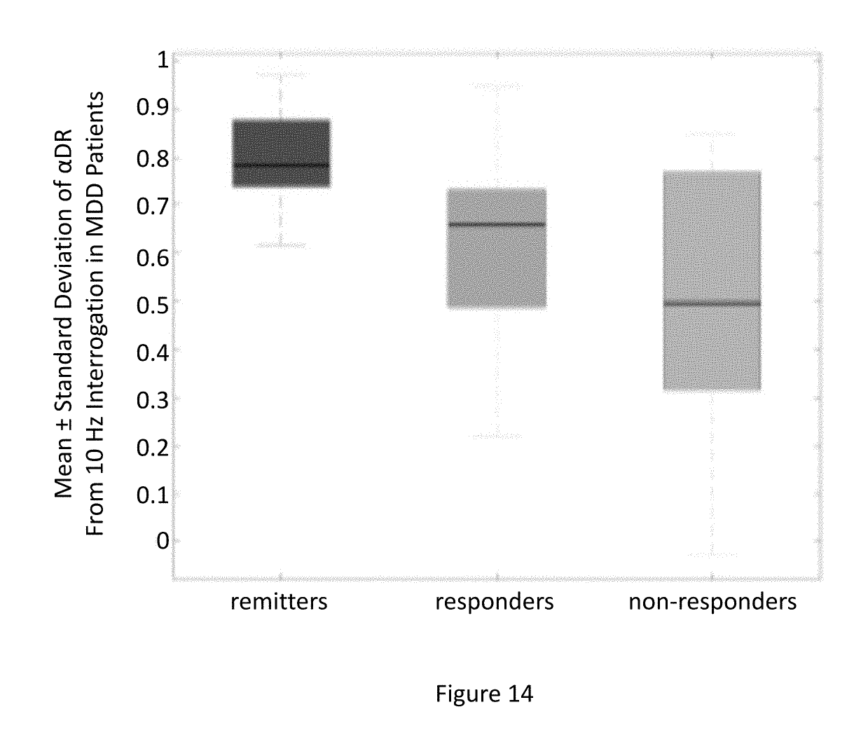

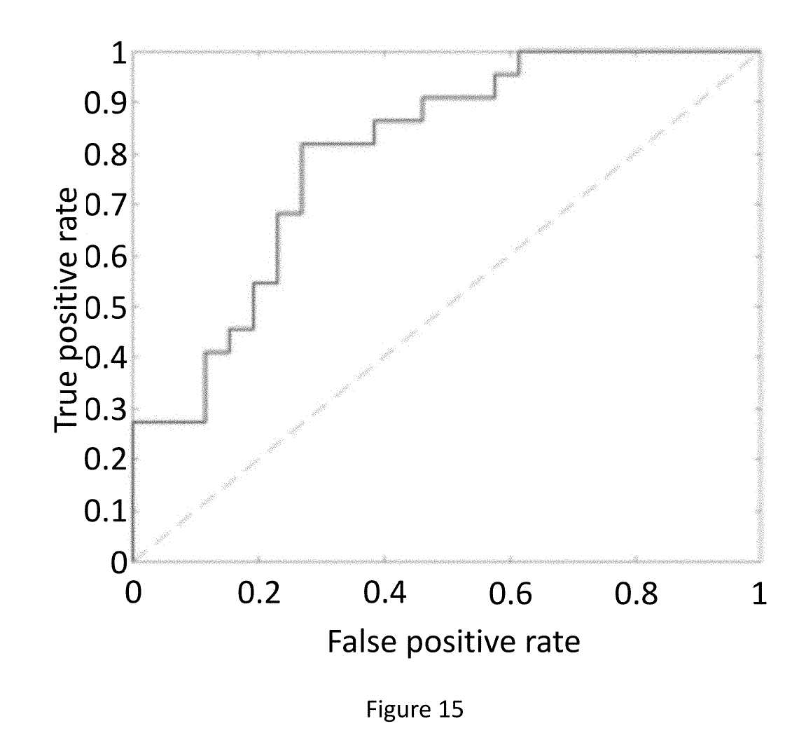

αDR during target interrogation is associated with response and remission. 52 subjects with moderately severe MDD were examined, all of whom received a single session of 10 Hz αDR target interrogation to left DLPFC prior to a course of rTMS treatment. Clinical symptoms over the course of treatment were assessed using the IDS-SR (Inventory of Depressive Symptomatology-Self-rated), with categorical outcome (response, remission) based upon the final IDS-SR score after treatment 30. Results showed average αDR across four electrode pairs (Fp1-Pz, Fpz-Pz, F3-Pz, and F5-Pz) from the initial target interrogation was significantly higher in remitters than in responders or non-responders (p=0.004) (FIG. 14). These same electrode pairs were used in a logistic regression analysis, which showed that a linear combination of these pairs classified remitters/responders vs. non-responders with an accuracy of 80% (p=0.0009) (FIG. 15).

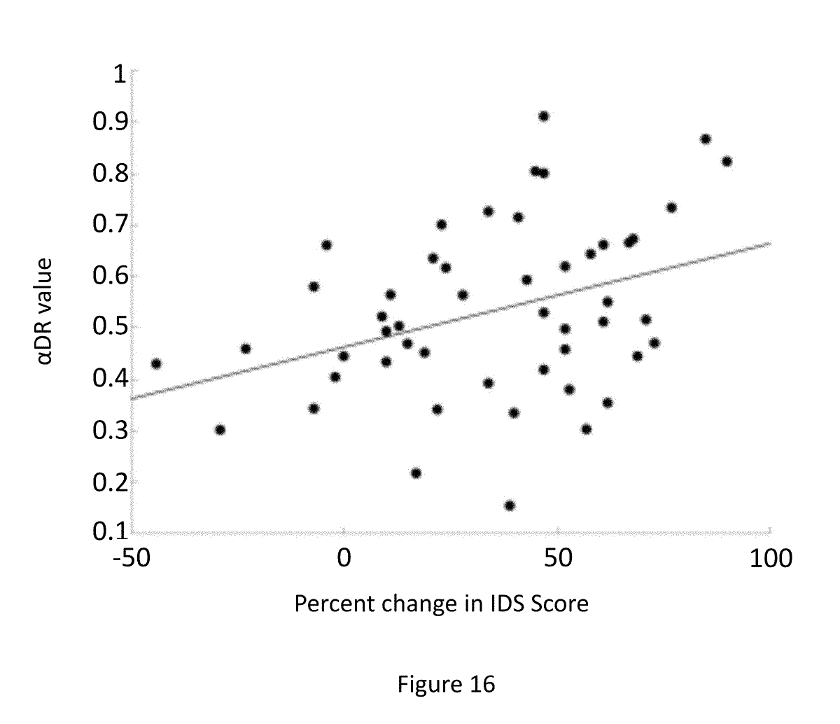

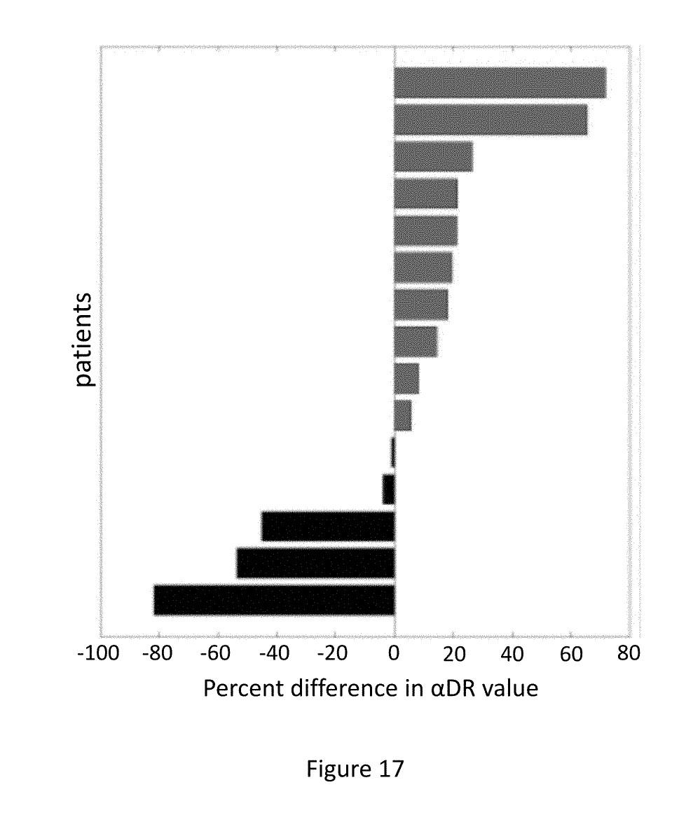

In addition to a relationship with categorical response, magnitude of αDR from the initial target interrogation is associated with degree of clinical improvement. In the same 52 patients examined above, initial magnitude of αDR across the four electrode pairs was significantly correlated with change in IDS score after 6 weeks (r=0.44, p=0.007) (FIG. 16).

Interrogation with IAF stimulation generates greater αDR than interrogation with 10 Hz stimulation for a significant proportion of subjects. In order to test the αDR target interrogation procedures proposed here, these methods were utilized to perform a separate pilot study comparing the effects of 10 Hz and IAF target interrogation on magnitude of αDR in 15 subjects with MDD. These subjects underwent target interrogation with both IAF and 10 Hz prior to rTMS treatment, following the procedures described below. The magnitude of αDR elicited by both 10 Hz and IAF interrogations were compared, and the percentage by which the magnitude of one form of interrogation exceeded the other was calculated. It was found that in 10 of 15 subjects (66%), IAF interrogation elicited a greater magnitude of αDR than did 10 Hz, with an average 27.3% greater magnitude (range 5.7-71.3%). There was no relationship between the order in which interrogation was administered and which interrogation was of larger magnitude. Three of four subjects with larger αDR at IAF who showed the largest percentage difference also had the greatest frequency difference between their IAF and 10 Hz. This finding demonstrates that it may be important to include the ±difference between IAF and 10 Hz as a covariate in analyses. The interrogation pulse frequency generating the largest αDR is expected to be best for guiding the selection of parameters to be for the subsequent six-week course of rTMS therapy.

Significant evidence is provided in support of the importance of: 1) examining different frequencies of stimulation delivered to left DLPFC; 2) focusing on the α frequency band of neural oscillations; 3) selecting each subject's IAF within the α band; 4) investigating the FCN circuit in subjects with MDD; 5) development of the novel αDR measure for dynamic network response; and, 6) using rTMS target interrogation at baseline to identify a stimulation frequency that may enhance rTMS treatment outcomes. The novel αDR measure is a highly innovative approach to target interrogation.

Two Phase Study

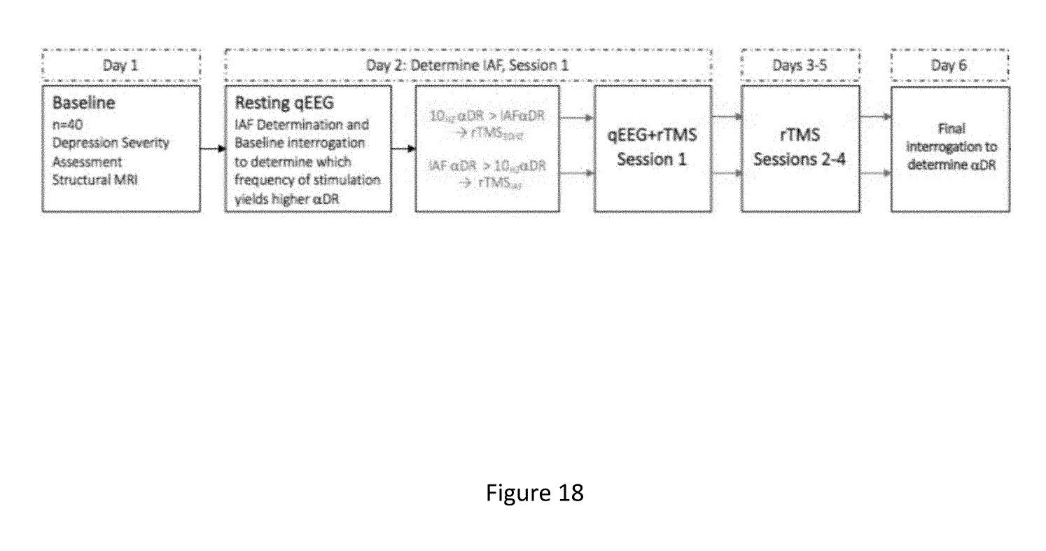

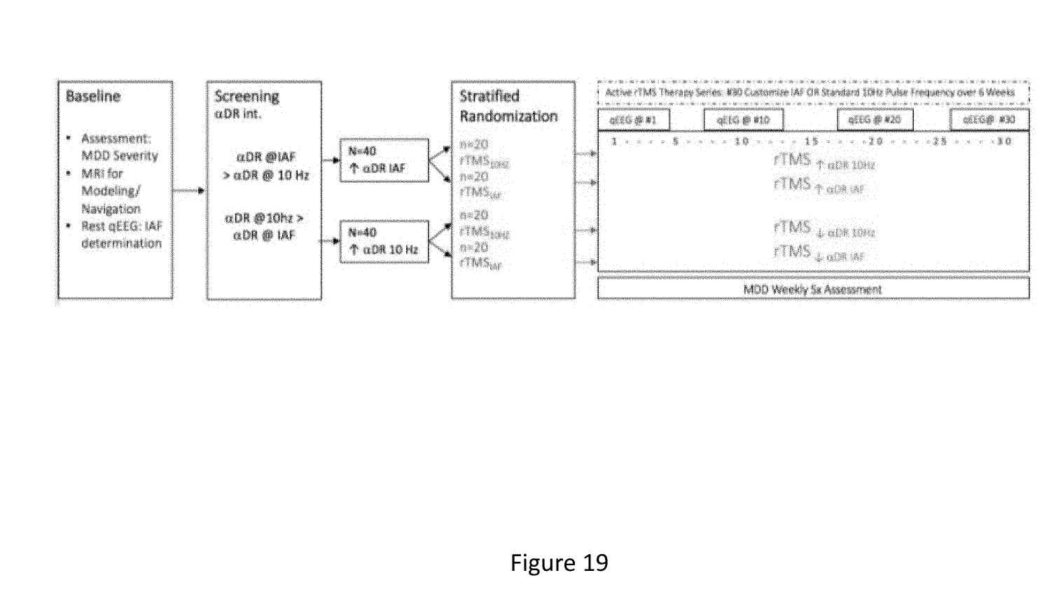

The first phase of the study enrolls 40 subjects with MDD (20 per site) in order to validate a dynamic response (αDR), a novel qEEG measure of target engagement (FIG. 18). αDR detects dynamic response of the frontoparietal control network (FCN) circuit to interrogation with rTMS test pulses at different frequencies. Data presented above indicate that αDR magnitude following interrogation indicates target engagement and is strongly correlated with the outcome of 30 sessions of 10 Hz rTMS treatment. In the R61 phase, MDD subjects (n=40) are first examined with baseline Mill for creating individual head models. These models are used to standardize spatial targeting and establish parameters for on- and off-target spread of rTMS stimulation. On Day 2, a standard (10 Hz, or rTMS_{10Hz}) and individualized (peak IAF, or rTMS_{IAF}) α stimulation frequencies are used as “doses” and will perform target interrogation at baseline to generate two αDR measures (determined at 10 Hz and IAF, respectively). Immediately after the second Day 2 interrogation session, it is determined which of the two pulse frequencies produced αDR of greater magnitude. Each subject has five sessions of daily rTMS_{10Hz} or rTMS_{IAF} treatment at the frequency that elicited the αDR measure of higher magnitude. On Day 6 (rTMS session 5), a final αDR target interrogation is performed to assess cumulative effects of multiple applications of rTMS at the assigned frequency.

The magnitude of αDR from both the Day 2 and Day 6 target interrogation sessions (at the same frequency) are examined for each subject. The proportion of subjects is determined for whom the rTMS_{IAF} dose produces αDR values that are at least 5% greater than the magnitude of αDR from rTMS_{10Hz} dose at both interrogation sessions. A consistent effect is expected for the acute and cumulative effects of target interrogation (i.e., αDR magnitude from the Day 2 and Day 6 interrogations sessions, respectively). It is expected that both acute and cumulative magnitude of αDR determined at IAF will be at least 5% greater than the magnitude of αDR determined at 10 Hz in at least 55% of subjects (thresholds based upon pilot data presented above and discussed further below). Fulfillment of this criterion indicates that the αDR measure can identify a sufficiently large group of subjects with a larger magnitude of response to the IAF dose.

The second phase of the study enrolls 80 MDD subjects (40 per site) with the aims of: 1) confirming target engagement, and 2) conducting an initial test of the superiority of an individualized medicine approach to rTMS based upon frequency selection. All subjects undergo a baseline clinical screening and assessment as well as a qEEG, which are used to determine their IAF. Subjects also will be screened with a baseline αDR target interrogation using rTMS_{IAF} and rTMS_{10Hz}, which allows for the classification of individual subjects based upon which interrogation frequency produces the higher magnitude αDR. 80 subjects are enrolled in two groups (40 with αDR in response to 10 Hz>αDR in response to IAF interrogation, and 40 with αDR in response to IAF>αDR in response to 10 Hz interrogation).

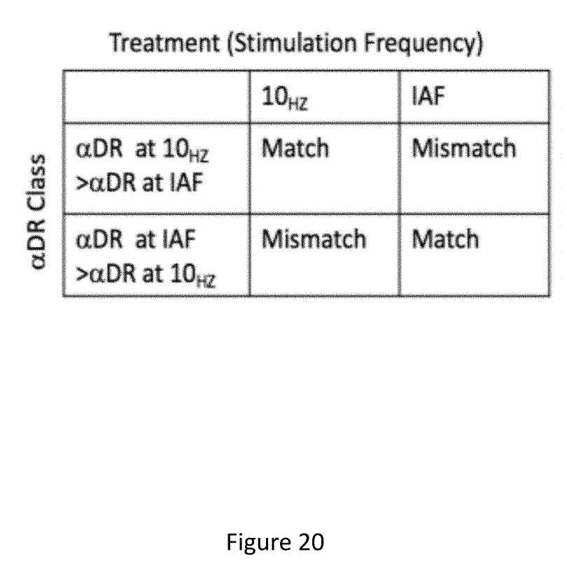

A six-week double-blind treatment trial is conducted using stratified randomization to assign subjects to rTMS_{IAF} vs. rTMS_{10Hz}. It is expected that subjects whose αDR status matches subsequent treatment (i.e., αDR at IAF>αDR at 10 Hz and treated with rTMS_{IAF}, or αDR at 10 Hz>αDR at IAF and treated with rTMS_{10Hz}) will have a better rTMS clinical response than those treated in a mismatch condition. Stratified randomization based upon αDR status therefore is used to assign subjects in equal numbers to matched and mismatched MM-guided rTMS_{10Hz} or rTMS_{IAF} treatment (FIG. 20). Subjects receive 30 rTMS treatments at the assigned stimulation frequency with weekly mood assessments, and the primary endpoint following six weeks of treatment. αDR target interrogation is performed at the assigned treatment frequency at baseline and after treatments 10, 20, and 30 in order to assess the cumulative effects of treatment on αDR. This phase of the study achieves two aims: (1) confirm target engagement—magnitude of αDR index measured at baseline and serially over 6 weeks of rTMS treatments is expected to be significantly correlated with degree of clinical improvement and, in a model predicting degree of clinical improvement, there is expected to be a significant interaction between treatment condition matching (rTMS_{matched} vs. rTMS_{mismatched}) and αDR value; (2) test the superiority of an individualized medicine approach—subjects receiving six weeks of rTMS_{matched} treatment are expected to show significantly greater clinical improvement than subjects receiving treatment than in the rTMS_{mismatched} condition.

Subject Selection

Individuals 18-65 years of age with a primary diagnosis of MDD determined by the MINI structured interview (Sheehan D V et al., J din psychiatry, 1998, 59.Suppl 20:2233), presenting with at least moderately severe depressive symptoms based upon a 17-item Hamilton Depression Rating Scale Score (Ham-D17) (Hamilton M, Journal of neurology, neurosurgery, and psychiatry, 1960, 23(1):56) greater than 17 and who currently are not taking antidepressant or other psychotropic medications are used for the study. Primary exclusions include another primary mood, anxiety, psychotic, or dementing illness, active substance abuse disorder, seizure disorder, or medical illness of sufficient severity to significantly affect brain function. Subjects with history of skull fracture are excluded because of breech rhythms in the EEG. Subjects must be able to undergo an MRI scan and, consistent with published consensus guidelines for clinical application of rTMS therapy (McClintock S M et al., The Journal of clinical psychiatry, 2017), rTMS-specific safety screening will be conducted and medical clearance for rTMS to include review of medical history and physical examination.

In addition to diagnostic interview with the MINI and severity assessment with the Ham-D17, depressive symptoms are assessed at baseline (and weekly during the R33 phase) with the Inventory of Depressive Symptomatology—Self-Rated Version (Trivedi M H et al., Psychological medicine, 2004, 34(1):73-82) as well as the Patient Health Questionnaire-9 (PHQ-9) (Lowe B et al., Medical care, 2004, 42(12):1194-1201), and the Montgomery Asberg Depression Rating Scale (MADRS) (Montgomery S A et al., The British journal of psychiatry, 1979, 134(4):382-389). All subjects are screened with aDR target interrogation at both 10 Hz and IAF in order to identify 40 subjects with αDR at IAF>αDR at 10 Hz, and 40 subjects with αDR at 10 Hz>αDR at IAF.

Following determination of their αDR category, eligible subjects are assigned to “matched” and “mismatched” treatment conditions using stratified randomization as shown in FIG. 20. Based upon the higher value of the two αDR measures, subjects are randomized to receive treatment at either their higher magnitude αDR frequency (match condition, or rTMS_{match}) or their lower αDR frequency (mismatch condition, or rTMS_{mismatch}). During 6 weeks of rTMS therapy, all treatments sessions are delivered at the same pulse frequency, according to cell assignment: (1) IAF match condition (αDR@IAF>αDR@10 Hz)−rTMS_{match} treatment@IAF; (2) IAF mismatch condition (αDR@IAF>αDR@10 Hz)−rTMS_{mismatch} treatment@10 Hz; (3) 10 Hz match condition (αDR@10 Hz>αDR@IAF)−rTMS_{match} treatment@10 Hz; (4) 10 Hz mismatch condition (αDR@10 Hz>αDR@IAF)−rTMS_{mismatch} treatment@IAF. Stratified randomization is performed within cells defined by gender and symptom severity to provide a degree of balance on key underlying characteristics that otherwise would have the potential to confound the interpretation of study findings. Specifically, there are eight strata for randomization defined by the 2×2×2 combinations of αDR, gender (male or female, and symptom severity (lower or higher).

EEG Recording

Data is recorded using the “eego mylab” TMS-compatible EEG system at a sampling rate of 1000 Hz (Advanced Neuro Technology [ANT]; Enschede, Netherlands). Electrodes are applied using the 64-electrode “WaveGuard” system with sintered Ag/AgCl electrodes mounted in an elastic cap and positioned according to the Extended 10-20 System with EOG electrodes above and below the left eye. The material and shape of the electrodes prevents current loops and is designed for minimal DC shifts and optimal stability of the incoming signal during TMS. Data are recorded using full-band EEG DC amplifiers that return to physiologic baseline signal level within 10 ms after the end of the TMS pulse. Filters are not applied during data acquisition, and recording is performed using a common average reference with impedance kept below 5 kΩ.

EEG Processing

Semi-automatic preprocessing for artifact detection is performed using the FASTER algorithm (Nolan H et al., Journal of neuroscience methods, 2010, 192(1):152-162). This EEGLAB toolbox removes muscle, heart, motion, ocular artifacts, and other noise using a multiple step procedure consisting of a) bandpass and notch filtering, b) ICA, c) rejection and/or interpolation of bad channels/epochs. The final step of preprocessing includes visual inspection of the data rejecting any remaining artifactual epochs.

Exploratory EEG Analysis

This study generates a rich dataset that is used to explore the potential value of other EEG measures. For example, in order to compare αDR to previously well-studied measures of local and distributed network connectivity, an exploratory coherence analysis is conducted. Coherence is commonly used as a measure of degree of relationship between two signals and has been previously successfully used for examination of oscillatory network activity in MDD (Leuchter A F et al., PLoS One, 2012, 7(2):e32508). Conventional coherence is compared against the αDR measure for better performance in predicting treatment outcome. Coherence assessment also helps to better characterize overall changes in network dynamics following rTMS treatment.

MRI Data Acquisition

Structural Mills are acquired using Siemens Prisma 3-Tesla scanners. Protocol consists of an echo planar T2-weighted gradient echo sequence (TR=800 ms; TE=36.6 ms; flip angle=52 degrees; matrix size=64×64; FOV 210 mm) with isometric 1.9 mm voxels, and a high-resolution T1-weighted volume or MPRAGE (TR=2300 ms, TE=2.29 ms, TI=900 ms, flip angle=8°, matrix size=192×192, FOV=240 mm, 160 slices), with 1 mm isometric voxels. Scans are used for induced field modeling as well as anatomical targeting during neuronavigation.

rTMS Motor Threshold Determination

All TMS procedures are performed with identical Magstim Rapid 2 stimulators and Magstim coils (Magstim, Whitland, South Wales, UK). Motor threshold (MT) determination is performed using EMG monitors integrated with the Magstim unit, with electrodes applied to the right hand. MT is defined as the minimum stimulus intensity (applied to the head over the left primary motor cortex area) that elicits a motor evoked potential (MEP) in the right abductor pollicis brevis (APB) or first dorsal interosseus (FDI) muscles for ≥50% of applied stimuli. This intensity, represented as % of maximum stimulator output, is used for calibration of the subsequent stimulation sessions.

rTMS Treatment

Subjects are seated in a semi-reclined position and before starting each TMS procedure, they are asked about current clinical status (physical and mental well-being), interim use of medications that impact cortical excitability, and, subsequent to first session, any adverse events associated with study procedures. Metal objects are removed from the head and neck area and earplugs are given for ear protection. Treatments consist of 3000 pulses delivered to the left DLPFC target at the stimulus intensity directed by e-field modeling for each subject up to 120% MT (to which subjects are accommodated in the first two treatment sessions). Treatments are administered in a single-blinded manner (subject blinding only) because of the inherent difficulty in blinding experienced technicians and physicians to pulse frequency. All pulses are delivered in 40 pulse bursts with adjustment of the intertrain interval (ITI) as needed to hold total treatment time constant. Data indicate that adjustment of the ITI in the range needed for this study will not have any significant effect on treatment efficacy or rTMS-induced cortical excitability (Cash R F H et al., Brain Stimulation, 2017, 10(3):630-636). Participants are observed during stimulation sessions to identify any events that may impact rTMS safety or antidepressant efficacy. Systematic assessment methods are used at both sites to generate consistent and detailed documentation of daily research procedures, adverse events, and relevant clinical information across the two sites.

Neuronavigation

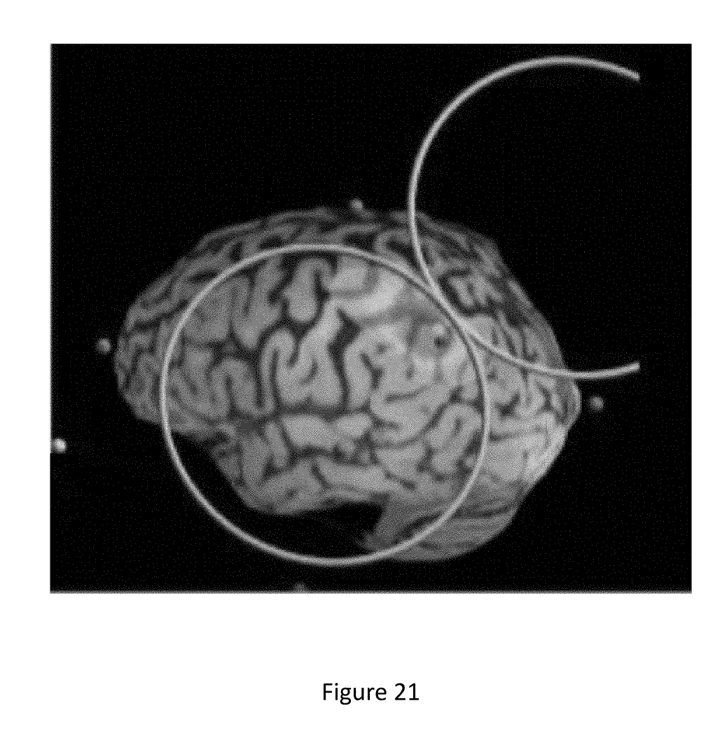

rTMS coil placement is performed with MM-guided frameless coil positioning using the ANT Visor2 system (ANT Neuro; Enschede, Netherlands). After loading each subject's raw MM images into the Visor software, the system allows visualization of the relationship among the stimulation target, associated scalp, and a 3D reconstruction of the subjects' brain (FIG. 21). The DLPFC stimulation target is defined in MNI coordinate space for each subject based upon their individual MRI scan, with coil position and angle maintained to be consistent with the e-field model parameters throughout the stimulation session.

Electric Field Modeling

All TMS stimulation procedures are performed based upon individual-level model derived from high-resolution T1 and T2 weighted MM scans performed prior to stimulation. simNIBS 2.0 (www.simnibs.org) is utilized to model current distributions with a finite element method (Windhoff M et al., Human brain mapping, 2013, 34(4):923-935; Thielscher A et al., Engineering in Medicine and Biology Society (EMBC), 2015 37th Annual International Conference of the IEEE; Madsen K H et al., Brain stimulation, 2015, 8(6):1205-1208). This model is based upon the magnetic dipole model for the Magstim 70-mm figure-of-eight coil and is based upon stimulator output defined relative to the peak current at 100% output, calculated as the root mean square of the peak current for a sinusoidal pulse duration of 300 ms. Stimulation is applied to the left DLPFC, defined as the central portion of the left middle frontal gyms (MNI coordinates—41, 23, 49). Stimulation intensity is adjusted to maintain selectivity in on-target vs. off-target stimulation. Temporal and spatial elements of stimulation are factored into the model including pulse direction and frequency as well as coil angle with regard to brain structure, and electrical conductivities are assigned to different tissue types with isotropic conductivity assumed (Windhoff M et al., Human brain mapping, 2013, 34(4):923-935; Wagner T et al., Cortex, 2009, 45(9):1025-1034).

Demonstration of Average Models

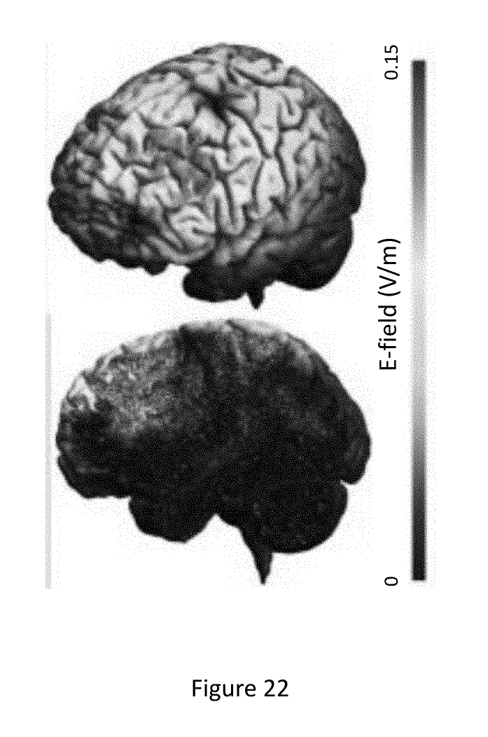

In preparation for this application, intervention was modeled in data obtained from 3 pilot subjects treated with 10 Hz stimulation applied to left DLPFC coordinates as specified above, using 4 second pulse train and 26 second ITI. Electric field intensity distribution was modeled using SimNIBS using two T1 and T2 weighted Mill data. The intensity was set to 50% of maximum system output, a typical level needed to achieve 120% of RMT in subjects. The results of the model are shown in FIG. 22, with intensity scaled to show all surface points where the electric field intensity was between 100-120% of the maximum current (Wagner T et al., Cortex, 2009, 45(9):1025-1034). This thresholding led to the determination of which neuroanatomic areas a therapeutic level of stimulation was delivered. These results indicate that the therapeutic level of stimulation is largely confined to the middle and inferior frontal gyri (Brodmann areas 46 and 9) that lie within the DLPFC, and that the ratio of target to non-target activity is high. Similar targeting techniques are used in the study to ensure accurate delivery of stimulation to the DLPFC.

Frequency-Based Modeling

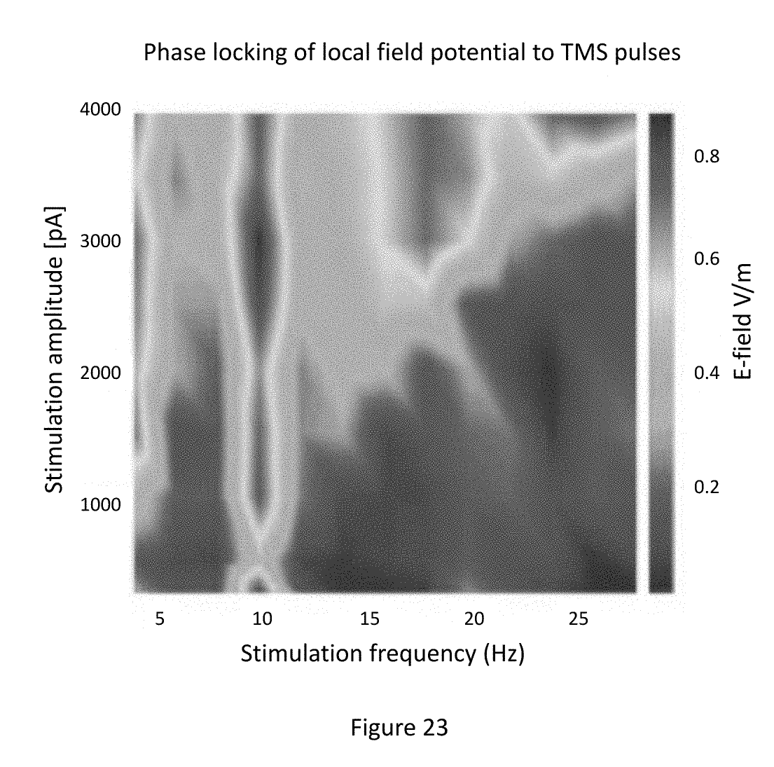

While the principles of electrical field modeling based upon current intensity are well established, the effects of different frequencies of stimulation are not well studied. It is unlikely that frequency adjustments in the narrow range proposed here will have significant differential effects on field spread. Because this is a novel approach, however, an exploratory investigation of the effects of frequency adjustments on the field will be taken via computer simulations of target engagement of a oscillations by rTMS pulse trains delivered at different frequencies, based upon phase-locking of the simulated local field potential and the stimulation waveform (TMS pulses). The simulated output of such a model is shown in FIG. 23. The model cortical network was tuned to exhibit an endogenous frequency in the α frequency range, with stimulation frequencies between 4 and 30 Hz evaluated for different stimulation amplitudes.

Individual a Frequency (IAF) Determination

Using artifact-free data obtained from EEG preprocessing, the frequency power spectrum is calculated using Welch's power spectral density estimate. Because the frequency resolution is dependent on the sampling frequency and data length used to compute the power spectra, the obtained frequency resolution is 0.25 Hz based on 4-second long artifact-free segments sampled at 1000 Hz. Power estimates for each frequency bin are expressed as the percentage of total power in the range 2-20 Hz. Each subject's IAF is determined by identifying the highest peak within the 7-13 Hz alpha range that surpasses a 95% confidence interval of the mean spectral power in the same range derived from a 2000-samples bootstrapped distribution. Using this alpha peak, a 4 Hz IAF band is created (IAF peak±2 Hz).

αDR Determination

The αDR measure captures the similarity in changes in power spectrum across two locations that are elicited by exposure to rTMS. This is computed in several steps. First, the pre-rTMS power spectrum is calculated (with 0.25 Hz resolution) for each EEG electrode of interest in the frontal region (Fp1, FPz, F3, F5) and the parietal region (Pz) using a common reference. Second, the relative power is calculated for a 4 Hz-wide band centered at the subject's IAF (±2 Hz), using total power from 2-20 Hz as the denominator for relative power normalization. Third, the same procedure to determine relative power in an IAF-centered band using qEEG signals recorded immediately after target interrogation with rTMS pulses. Fourth, the shift in spectral power is determined by subtracting the post-rTMS power value from the pre-rTMS value, separately in each prefrontal and parietal electrode, generating a frequency series of spectral shifts at each location. Fifth, similarity is assessed in shifts in the two regions by computing a Pearson bivariate correlation coefficient (R) between the prefrontal change and the parietal change, across all frequencies and for each electrode pairing (Eq 1). Finally, αDR is formed by averaging these correlations for the four anatomical pairs (Fp1-Pz, Fpz-Pz, F3-Pz, F5-Pz) (Eq 2). The average correlation across all target channels represents the final αDR value, ranging [−1,1], with 1 representing a high degree of positively correlated shifts (i.e., very similar shifts elected across the network) and −1 a negatively correlated dynamic response.

R_{FP}=corr((S_{t1,F}−S_{t0,F}),(S_{t1,P}−S_{t0,P})) (Eq.1)

S_{ti,Loc}=spectrum at IAF±2 Hz at frontal or parietal location
where tᵢ=t₀ for pre-rTMS and tᵢ=t₁ for post-rTMS

$$α DR = \frac{{\sum }_{i = 1}^{n} ( {R}_{FPi} )}{4} \qquad ( Eq 2 )$$

where n=4 for each of the 4 channel pairs

Target Interrogation Procedure

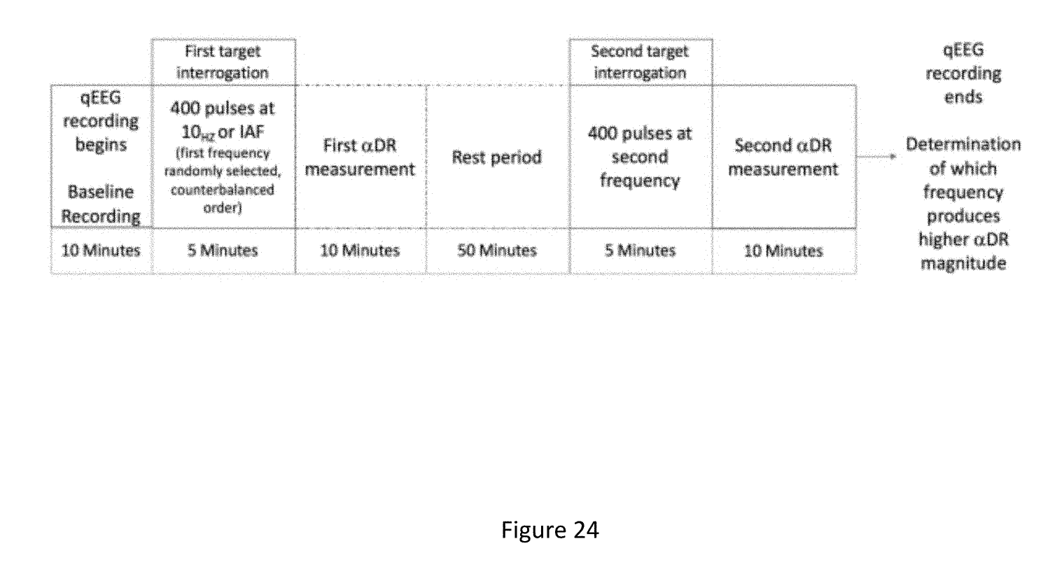

Target interrogation is performed using 10 trains of 40 pulses each (400 pulses) of rTMS stimulation administered at a specified a frequency (FIG. 24). Interrogations are performed at two frequencies (10 Hz and peak IAF) administered in counterbalanced order across subjects in order to control for order effects. Pilot data indicate that 200-800 pulses produce consistent αDR effects with no increase with greater numbers of pulses; here, 400 pulses are used to be toward the middle of this range. There is a 60 minute rest period between the interrogations. Existing literature indicates that the effects on excitability and oscillatory synchrony of even longer stimulation periods (900 pulses) end by 30 minutes (Bohotin V et al., Brain, 2002, 125(4):912-922; Hoogendam J M et al., Brain stimulation, 2010, 3(2):95-118; Thut G et al., Brain topography, 2010, 22(4):219), providing confidence that 60 minutes between interrogations prevent carry-over effects. Furthermore, no carry forward effects were detected in pilot data. 10 Hz interrogation is performed at a stimulation frequency of 10 Hz, and IAF interrogation is carried out at the peak IAF rounded up to the nearest 0.1 Hz value based upon the capability of the Magstim device to deliver stimulation in 0.1 Hz increments. The only exception to this procedure will be if a subject's IAF is determined to be exactly 10 Hz (in pilot data, this occurred in 4/52 or ˜8% subjects). If a subject's IAF is determined to be at 10 Hz, the interrogations will be at 10 Hz and at 11 Hz (10+1 Hz), consistent with the observations of enhanced cognitive performance following IAF+1 stimulation (Klimesch W et al., European Journal of Neuroscience, 2013, 17(5):1129-1133).

### Example 2: 112 Subjects with MDD Treated with rTMS

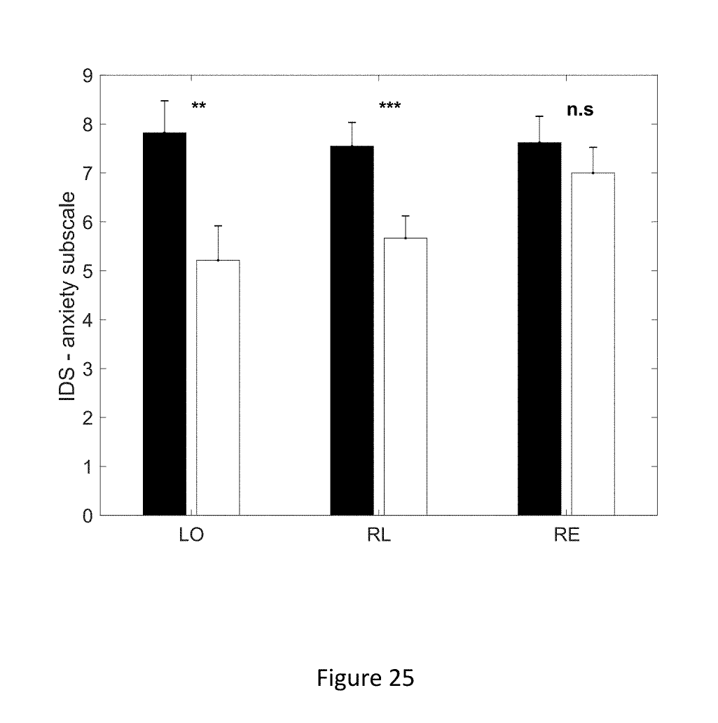

The present study investigates the relationship between the change in alpha frequency (7-13 Hz) oscillations during the first rTMS session and clinical response to rTMS treatment in a cohort of 112 subjects with MDD. All subjects received 30 sessions of 10 Hz rTMS treatment delivered to left DLPFC. Subjects who showed early and/or sustained improvement in response to 10 Hz rTMS to left DLPFC were maintained on this treatment alone (n=28), and those who showed improvement that was not incremental or sustained after treatment 15 had concomitant treatment with 1 Hz rTMS administered to right DLPFC in the second half of the treatment course (n=42). Subjects who showed no significant improvement in depressive or anxiety symptoms and/or a clear worsening in response to 10 Hz rTMS to left DLPFC had concomitant right-sided treatment added before treatment 15 (n=42) (FIG. 25). Mood and anxiety symptoms were monitored weekly during treatment with the Inventory of Depressive Symptomatology (IDS) as well as Clinical Global Impression of Improvement (CGI-I).

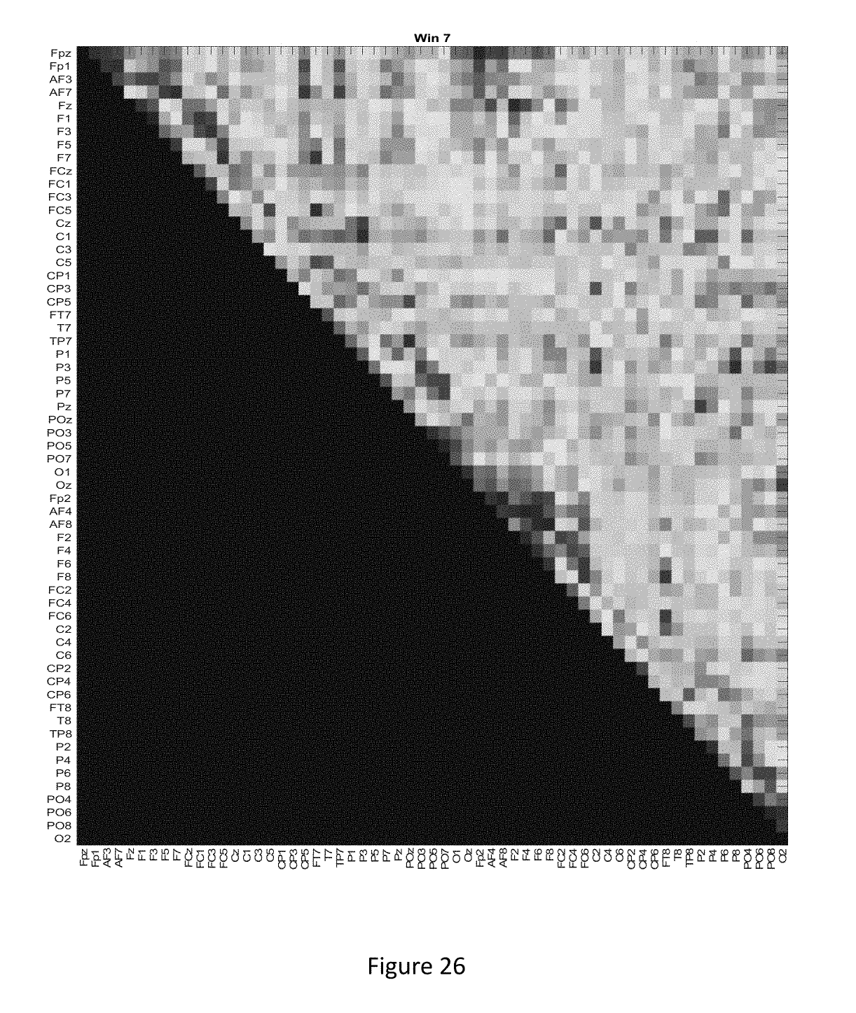

The independent measure was the dynamic response of oscillations in each subject's individual alpha frequency band (alpha dynamic response, or αDR). αDR was calculated between electrode seeds in the left and right prefrontal regions and all other electrode locations. Prefrontal seeds were chosen because these represented the site of stimulation in the first treatment session (left) and the homologous contralateral region (right). αDR therefore was calculated for a total of 846 electrode pairs (FIG. 26).

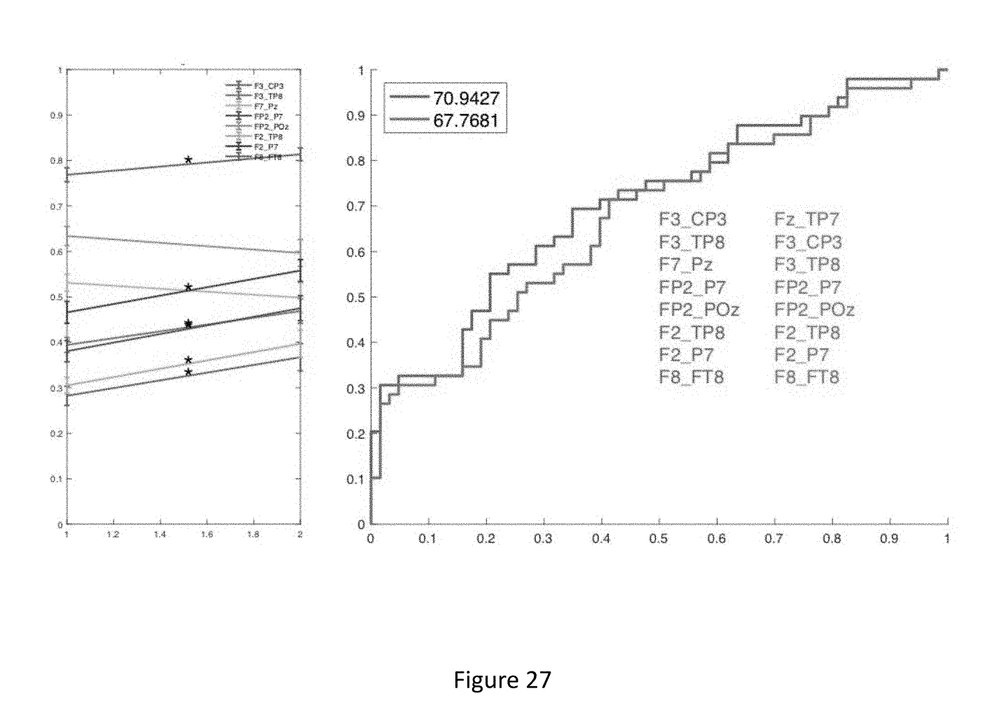

For the overall sample (n=112), higher αDR was significantly higher overall in responders than non-responders. Significant differences were seen for connections between left and right prefrontal cortices and both ipsilateral and contralateral temporal and parietal cortices bilaterally. A complex pattern of eight connections, six showing higher and two showing lower dynamic response in responders than in non-responders, accurately classified 67-70% of subjects into responder and non-responder categories (FIG. 27).

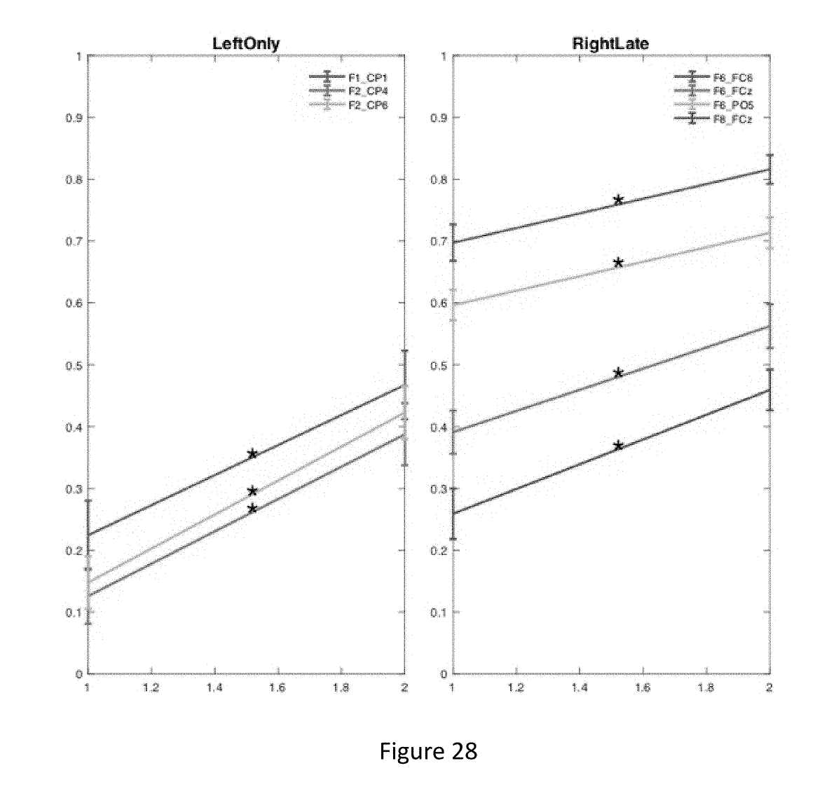

When the three treatment groups were examined separately, a more consistent pattern of relationship between dynamic response and treatment outcome emerged (FIG. 28). Those subjects who showed the clearest response to 10 Hz stimulation of left DLPFC either alone (n=28) or in combination with 1 Hz stimulation of right DLPFC added late in the course of treatment (n=42) showed a pattern of significantly higher αDR in responders than in non-responders for all left and right prefrontal seeds. Responders were distinguished from non-responders, however, by uniquely showing significantly greater αDR between right prefrontal seeds and other regions within the right prefrontal, right parietal, or left parietal regions. Subjects who responded to 10 Hz left DLPFC stimulation alone had significantly higher αDR between an electrode located over the right superior frontal gyms (F2, BA6) and the right superior temporal gyms (CP6, BA40) than those who did not respond. This single variable accurately identified 91% of responders (FIG. 28, left). Subjects who responded to left DLPFC stimulation with late addition of right DLPFC stimulation showed higher αDR between electrodes overlying the right middle frontal gyms (right DLPFC, F6 and F8, BAs 9/45/46/47) and the superior frontal gyms (FCz, BA6) as well as the left middle occipital gyms (PO5, BA19) (FIG. 28, left). This combination of three electrode pairs predicted response with 82% accuracy. These findings suggest that while left DLPFC stimulation affects the dynamic response of cortex immediately underneath the stimulating magnet, it is the extent to which this effect elicits high dynamic response in cortex contralateral to stimulation that predicts therapeutic benefit.

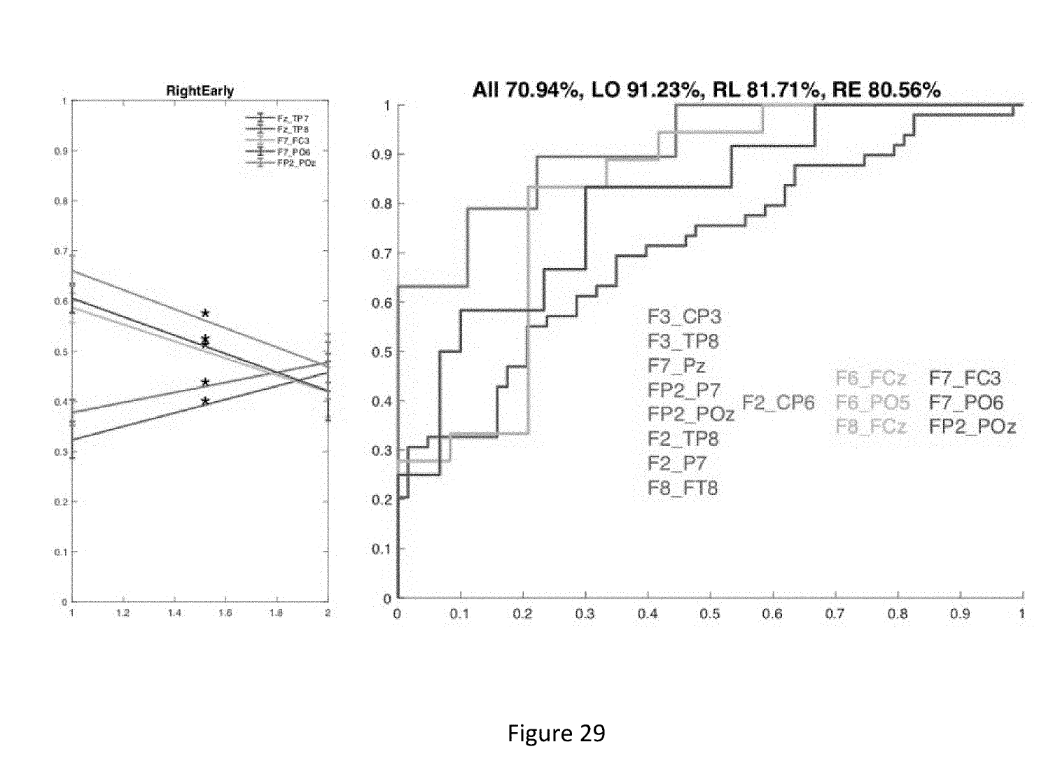

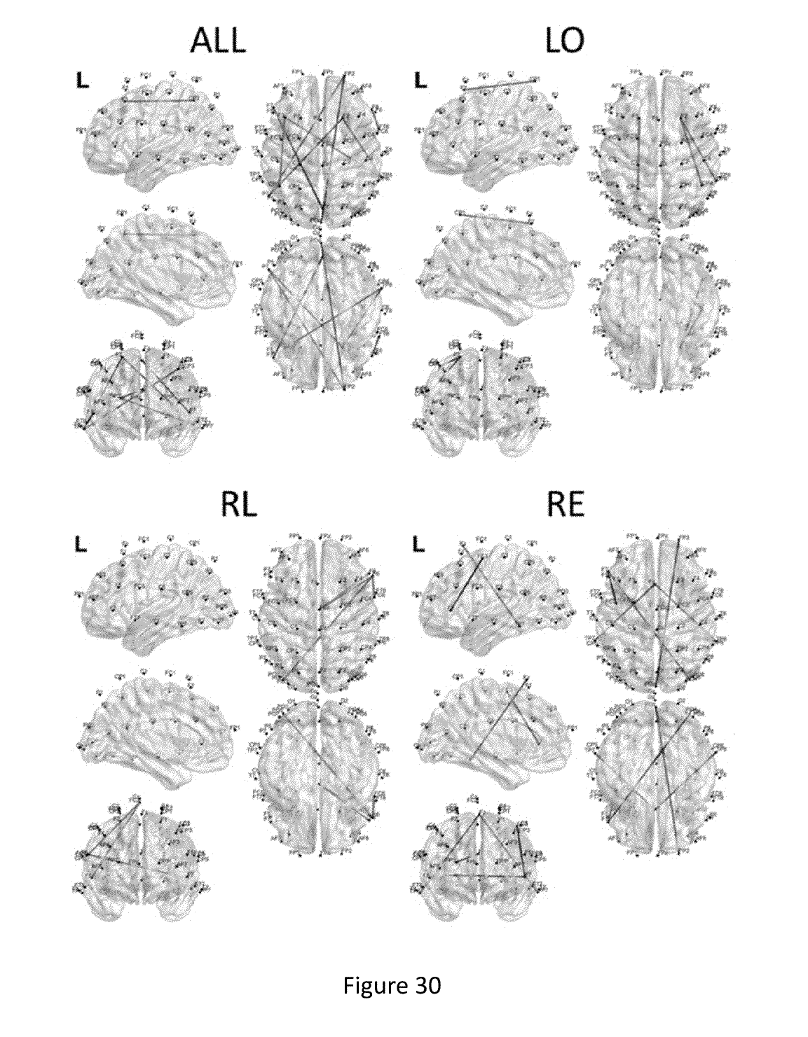

For subjects who received left DLPFC 10 Hz stimulation combined with right DLPFC stimulation for the majority of their treatment (n=42), a different pattern emerged (FIG. 29). Subjects who responded to this form of stimulation showed lower aDR between electrodes located over left and right lateral prefrontal cortex and those in prefrontal cortex or left and right parieto-occipital cortex. Specifically, responders had lower αDR than non-responders between electrodes over the left inferior frontal gyms (DLPFC, F7, BA 45/46/47) and right superior frontal gyms (Fp2, BA10) and electrodes over right middle occipital gyms (PO6, BA 19), the cuneus (POz, BA19), and the left middle frontal gyms (FC3, BA6). Responders did show higher αDR than non-responders between an electrode located over the superior frontal gyms (Fz, BA6) and electrodes located over the middle temporal gyms bilaterally (TP7 and TP8, BA21). The combination of five electrode pairs showing a mixture of lower and high αDR values predicted response with 89% accuracy.

rTMS is a treatment that is believed to reset brain functional network connectivity, and the effects of rTMS stimulation are known to spread through brain functional networks even with the first treatment session. The present findings suggest that both the topographic pattern and the direction of change in oscillatory synchrony in the first rTMS treatment session with 10 Hz stimulation of left DLPFC are important predictors of outcome. High dynamic oscillatory response (αDR) seen in functional connections of lateral prefrontal cortex contralateral to the site of stimulation are strongly associated with response after 30 sessions of treatment. Conversely, low dynamic oscillatory response seen in connections of lateral prefrontal cortex both ipsilateral and contralateral to site of stimulation are uniquely associated with lack of improvement and poor treatment outcome.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A non-transitory computer-readable medium with instructions stored thereon, that when executed by a processor, determines a correlation coefficient of the shift in pre- and post-transcranial magnetic stimulation (TMS) treatment between a pair of EEG electrodes positioned on at least two sites of a subject's brain, by performing the steps comprising:
measuring a subject's pre-rTMS power spectrum at an EEG electrode of interest;
calculating relative power from the subject's pre-rTMS power spectrum measurements at the EEG electrode of interest;
measuring a subject's post-rTMS power spectrum at the EEG electrode of interest;
calculating a relative power from the subject's post-rTMS power spectrum measurements at the EEG electrode of interest;
obtaining a correlation coefficient R
R=corr((S_{t1,F}−S_{t0,F}),(S_{t1,S}−S_{t0,S})) (Eq.1)
wherein S_{ti,Loc}=relative power of the spectrum measurements; tᵢ=t₀ for pre-rTMS; tᵢ=t₁ for post-rTMS; Loc=F for first brain site; and Loc=S for second brain site.

2. The non-transitory computer-readable medium of claim 1, wherein the stored instructions further comprise the step of determining a dynamic response (DR) value from the correlation coefficients of a selection of electroencephalography (EEG) electrode pairs, wherein the DR value is defined according to:
$$DR = \frac{{\sum }_{i = 1}^{n} ( {R}_{i} )}{n} \qquad ( Eq 2 )$$
wherein n=the number of selected EEG electrode pairs.

3. The non-transitory computer-readable medium of claim 1, wherein the power spectrum is measured in the alpha band, the beta band, the gamma band, the delta band, or the theta band.

4. The non-transitory computer-readable medium of claim 2, wherein the at least two sites of the subject's brain are selected from the group consisting of the left or right side of: the frontal lobe, the central lobe, the temporal lobe, the parietal lobe, the occipital lobe, the motor cortex, the premotor cortex, the prefrontal cortex, the somatosensory cortex, the posterior parietal cortex, the visual cortex, the auditory cortex, the temporal cortex, the frontal gyms, the postcentral gyms, the lateral occipital gyms, the temporal gyms, the Brodmann areas, the cuneus, the precuneus, and combinations thereof.

5. The non-transitory computer-readable medium of claim 4, wherein the two sites of the subject's brain are the frontal lobe and the parietal lobe.

6. The non-transitory computer-readable medium of claim 2, wherein the at least two sites of the subject's brain form part of a brain network selected from the group consisting of: the frontoparietal control network (FCN); the default mode network (DMN); the salience network (SN); the dorsal attention network (DAN); the ventral attention network (VAN); the basal ganglia network (BGN); the limbic network (LN); the somatomotor network (SMN); the visual network (VN); the frontoparietal network (FPN); the anterior insula network (AIN); the executive control network (ECN); the executive attention network (EAN); the medial visual network (MVN); the lateral visual network (LVN); the cerebellar network (CBLN); the auditory network (AN); the task positive network (TPN); and the self-referential network (SRN).

7. The non-transitory computer-readable medium of claim 2, wherein the dynamic response is the alpha dynamic response (αDR), the beta dynamic response (βDR), the gamma dynamic response (γDR), the delta dynamic response (ΔDR), or the theta dynamic response (θDR).

8. The non-transitory computer-readable medium of claim 7, wherein the power spectrum is measured at a peak individual alpha frequency (IAF) value in the range between 2 and 20 Hz.

9. The non-transitory computer-readable medium of claim 8, wherein the power spectrum is measured between an IAF band between 2 Hz below the IAF value and 2 Hz above the IAF value.

10. The non-transitory computer-readable medium of claim 8, wherein the subject's pre-TMS power spectrum at the EEG electrode of interest is measured immediately before the administration of a TMS treatment session.

11. The non-transitory computer-readable medium of claim 8, wherein the subject's post-TMS power spectrum at the EEG electrode of interest is measured one minute after administration of a TMS treatment session.

12. The non-transitory computer-readable medium of claim 2, wherein the selection of EEG electrode pairs is the four EEG electrode pairs: Fp1-Pz, Fpz-Pz, F3-Pz, and F5-Pz.

13. The non-transitory computer-readable medium of claim 2, wherein the determined DR value is between −1 and 1, such that a value closer to 1 indicates the subject has greater responsiveness to the rTMS treatment and a value closer to −1 indicates the subject has lesser responsiveness to the rTMS treatment.

14. A method of monitoring treatment of a neuropsychiatric disorder in a subject, comprising the steps of:
recording pretreatment quantitative electroencephalogram (qEEG) measurements of the subject as a function of relative power over a frequency range between 2 and 20 Hz;
treating the subject;
recording posttreatment qEEG measurements of the subject as a function of relative power over a frequency range between 2 and 20 Hz; and
rating the effectiveness of the treatment based on the change in the frequency of peak relative power between pretreatment and posttreatment qEEG measurements.

15. The method of claim 14, wherein the change between pretreatment and posttreatment qEEG measurements shows an increased and narrowed mean frequency primary peak and the emergence of a higher frequency secondary peak, indicating the treatment is highly effective in treating the subject.

16. The method of claim 14, wherein the change between pretreatment and posttreatment qEEG measurements shows a narrowed mean frequency primary peak and the emergence of one or more higher frequency secondary peaks, indicating the treatment is moderately effective in treating the subject.

17. The method of claim 14, wherein the change between pretreatment and posttreatment qEEG measurements shows a decreased mean frequency primary peak and the emergence of one or more higher frequency secondary peaks, indicating the treatment is slightly effective in treating the subject.

18. The method of claim 14, wherein the change between pretreatment and posttreatment qEEG measurements shows little to no change in the mean frequency primary peaks and secondary peaks, indicating the treatment is not very effective in treating the subject.

19. The method of claim 14, wherein the neuropsychiatric disorder is selected from the group consisting of: major depressive disorder (MDD), anxiety, post-traumatic stress disorder (PTSD), obsessive compulsive disorder (OCD), and Parkinson's disease.

20. The method of claim 14, wherein the treatment is selected from the group consisting of: repetitive transcranial magnetic stimulation (rTMS), deep brain stimulation, and transcranial direct current stimulation.

21. The method of claim 14, wherein the pretreatment and posttreatment qEEG measurements are recorded between 7.5 and 14 Hz.
